(19) United States
(12) Patent Application Publication
(10) Pub. No.: US 2025/0073247 A1
(43) Pub. Date: Mar. 6, 2025

(54) MURICHOLIC ACIDS AND DERIVATIVES FOR CHOLESTASIS TREATMENT

(71) Applicant: The Board of Regents of the University of Oklahoma, Norman, OK (US)

(72) Inventor: Tiangang LI, Edmond, OK (US)

(21) Appl. No.: 18/822,964

(22) Filed: Sep. 3, 2024

Related U.S. Application Data
(60) Provisional application No. 63/580,735, filed on Sep. 6, 2023.

(51) Int. Cl. A61K 31/575; A61K 47/18; A61K 47/20; A61K 47/54; A61P 1/16

(52) U.S. Cl. CPC A61K 31/575; A61K 47/183; A61K 47/20; A61K 47/54; A61P 1/16

(57) ABSTRACT

A method of treating a cholestasis liver condition in a subject in need of such therapy, comprising administering to the subject a muricholic acid (MCA), such as α-MCA, β-MCA, ω-MCA, or a glycine- or taurine-conjugated MCA, such as a glycine-conjugated α-MCA, a glycine-conjugated β-MCA, a glycine-conjugated ω-MCA, a taurine-conjugated α-MCA, a taurine-conjugated β-MCA, a taurine-conjugated ω-MCA, or a pharmaceutically-acceptable salt of any of the above.

### CROSS REFERENCE TO RELATED APPLICATIONS

This application claims benefit under 35 USC § 119(e) of U.S. Provisional Application Ser. No. 63/580,735 filed Sep. 6, 2023. The entire contents of the above-referenced patent application(s) are hereby expressly incorporated herein by reference.

### STATEMENT OF GOVERNMENT SUPPORT

This invention was made with government support under National Institutes of Health Grant No. DK117965. The government has certain rights in the invention.

### REFERENCE TO SEQUENCE LISTING SUBMITTED ELECTRONICALLY

The instant application contains, as a separate part of the present disclosure, a Sequence Listing which has been submitted via Patent Center in computer readable form as an XML file. The Sequence Listing, created Sep. 3, 2024, is named “5864.170 Sequence Listing.xml” and is 22,123 bytes in size. The entire contents of the Sequence Listing are hereby incorporated herein by reference.

### BACKGROUND

Bile acids are synthesized from cholesterol in hepatocytes and act as physiologic detergents to emulsify dietary lipids and signaling molecules to regulate various aspects of physiology in the enterohepatic system. Cholesterol 7α-hydroxylase (CYP7A1) mediates the rate-limiting step in hepatic primary bile acid synthesis. Bile acids are released into small intestine in response to food intake and re-absorbed in the terminal ileum to be transported back to the liver. In the gut, bacterial enzymes can further modify bile acids via deconjugation, dihydroxylation, and epimerization reactions to convert primary bile acids into secondary bile acids, which are either excreted or reabsorbed to enter the enterohepatic circulation. Therefore, the bile acid pool is a mixture of primary and secondary bile acids with different hydrophobicity and signaling properties. Bile acids serve as endogenous ligands for nuclear receptor farnesoid x receptor (FXR) and Takeda G protein-coupled receptor 5 (TGR5), which play important roles in regulating cholesterol and bile acid homeostasis, nutrient metabolism, immune response, and cell proliferation.

Dysregulation of bile acid homeostasis has been causally linked to many human metabolic and inflammatory diseases. Cholestasis is a major form of liver disease caused by impaired bile flow out of the liver, and the resulting intrahepatic bile acid accumulation leads to parenchymal and bile duct injury. Genetic defects in liver transporters and inflammation and autoimmune-mediated destruction of bile ducts are some of the common causes of cholestasis in humans. For most of the past 3 decades, ursodeoxycholic acid (UDCA), a hydrophilic bile acid, has been the only available treatment for several forms of cholestasis in humans. Mechanistic studies suggest that UDCA provides multi-folds hepatobiliary protection by decreasing bile acid pool hydrophobicity, promoting choleresis, and inhibiting inflammation. FXR agonist obeticholic acid (OCA) has been approved as a second treatment, either in combination with UDCA or as a monotherapy, for primary biliary cholangitis, which is caused by autoimmune destruction of intrahepatic small bile ducts. OCA has not been approved for treating other forms of cholestasis. A common adverse effect of OCA is pruritus.

Currently, therapeutic options are still limited for patients who do not adequately respond to these available treatments. Fibroblast growth factor 19 (FGF19) analogue and inhibitors of the gut bile acid uptake transporter apical sodium-dependent bile acid transporter (ASBT) have been investigated as potential new therapeutics for cholestasis.

It is known that bile acids are conjugated to either glycine or taurine in humans, while bile acids in mice are almost exclusively conjugated to taurine. Another notable difference in the human and murine bile acid metabolism is the bile acid pool composition. Although primary bile acid synthesis pathway is well conserved, the majority of chenodeoxycholic acid (CDCA) is subsequently metabolized to hydrophilic muricholic acids by CYP2C70 in mice. As a result, mouse bile acid pool contains primary bile acids taurine-conjugated cholic acid (T-CA) and taurine-conjugated muricholic acid (T-MCA) while human bile acid pool contains primary bile acids glycine-conjugated cholic acid (G-CA) and glycine-conjugated chenodeoxycholic acid (G-CDCA) as well as T-CA and taurine-conjugated chenodeoxycholic acid (T-CDCA). This difference renders mouse bile acid pool more hydrophilic and, if accumulated in liver due to cholestasis, less toxic than human bile acid pool. In addition, MCAs have been demonstrated to act as FXR antagonist, while the abundant bile acids in humans CA, CDCA and DCA all act as FXR agonists. Alteration of CA to MCA ratio in mice has been shown to significantly impact both the physiologic detergent function and signaling property of the bile acid pool, while alteration of CDCA to CA ratio is expected to have minimal impact on bile acid pool hydrophobicity or signaling property in humans. It has also been shown that treating mice with glycine-conjugated β-MCA (G-β-MCA), which is not an abundant naturally occurring bile acid in mice, antagonizes intestine FXR signaling and improves metabolic homeostasis in mice. Recently, Cyp2c70 knockout mice have been generated. The bile acid pool of the Cyp2c70 KO mice contains primarily hydrophobic CDCA and CA, and these mice developed hydrophobic bile acid-induced hepatobiliary injury. Several studies have utilized Cyp2c70 KO mice as a model to investigate the efficacy of various therapeutic agents against human like bile acid pool-induced hepatobiliary injury.

### BRIEF DESCRIPTION OF THE DRAWINGS

The patent or application file contains at least one drawing executed in color. Copies of this patent or patent application publication with color drawings will be provided by the Office upon request and payment of the necessary fec.

FIG. 1 shows body weight of mice (upper left panel), liver weight (upper right panel), and liver weight (LW) to body weight ratio (lower panel) after G-β-MCA treatment. Treatment alleviates liver ductal reaction and fibrosis in Cyp2c70 KO mice. Male Cyp2c70 KO mice at 8 weeks of age were fed a G-β-MCA containing chow diet for 5 weeks. The control mice were age and sex matched Cyp2c70 KO mice fed the same chow diet for 5 weeks.

FIG. 2 shows serum aspartate amino transferase (AST) concentration (upper panel) and alanine aminotransferase (ALT) concentration (lower panel) at the end of the treatment with G-β-MCA. Treatment alleviates liver ductal reaction and fibrosis in Cyp2c70 KO mice. Male Cyp2c70 KO mice at 8 weeks of age were fed a G-β-MCA containing chow diet for 5 weeks. The control mice were age and sex matched Cyp2c70 KO mice fed the same chow diet for 5 weeks.

FIG. 3 shows representative images of H&E stain, immunohistochemistry of F4/80 stain and CK19 stain (upper panel) and the CK19 positive area which was quantified by ImageJ Fiji software after G-β-MCA treatment. Treatment alleviates liver ductal reaction and fibrosis in Cyp2c70 KO mice. Male Cyp2c70 KO mice at 8 weeks of age were fed a G-β-MCA containing chow diet for 5 weeks. The control mice were age and sex matched Cyp2c70 KO mice fed the same chow diet for 5 weeks. Scale bar=250 μm.

FIG. 4 shows a representative Sirius Red Stain (upper panel), Sirius Red positive area per view quantified with ImageJ Fiji Software (lower left panel), and Ishak fibrosis score (lower right panel) after G-β-MCA treatment. Treatment alleviates liver ductal reaction and fibrosis in Cyp2c70 KO mice. Male Cyp2c70 KO mice at 8 weeks of age were fed a G-β-MCA containing chow diet for 5 weeks. The control mice were age and sex matched Cyp2c70 KO mice fed the same chow diet for 5 weeks. Scale bar=250 μm.

FIG. 5 shows effect of G-β-MCA treatment on liver mRNA expression of TNFalpha (upper panel), liver IL1beta (center panel), and liver IL-6 (lower panel). Male Cyp2c70 KO mice at 8 weeks of age were fed a G-β-MCA containing chow diet for 5 weeks. The control mice were age and sex matched Cyp2c70 KO mice fed the same chow diet for 5 weeks. Mice were fasted for 6 hours, from 9:00 am-3:00 μm, and then euthanized. A-C. show real-time PCR measurements of liver mRNA expression. Relative liver mRNA expression is expressed with the control set as “1.” n=11-13. All results are expressed as mean±SEM.

FIG. 6 shows that G-β-MCA treatment effects on mRNA expression of liver MCP1, liver COL1A1, and liver TIMP1 in Cyp2c70 KO mice. Male Cyp2c70 KO mice at 8 weeks of age were fed a G-β-MCA containing chow diet for 5 weeks. The control mice were age and sex matched Cyp2c70 KO mice fed the same chow diet for 5 weeks. Mice were fasted for 6 hours, from 9:00 am-3:00 μm, and then euthanized. Results are real-time PCR measurements of liver mRNA expression. Relative liver mRNA expression is expressed with the control set as “1.” n=11-13. All results are expressed as mean±SEM.

FIG. 7 shows that G-β-MCA treatment restores gut barrier integrity in Cyp2c70 KO mice. Male Cyp2c70 KO mice at 8 weeks of age were fed a G-β-MCA containing chow diet for 5 weeks. The control mice were age and sex matched Cyp2c70 KO mice fed the same chow diet for 5 weeks. Mice were fasted for 6 hours, from 9:00 am-3:00 μm, and then euthanized. Upper panel shows representative images of colon immunohistochemistry stain of ZO-1 from three mice per group. Scale bar=250 μm. In lower panel, male Cyp2c70 KO mice at 8 weeks of age were fed G-β-MCA containing chow diet for 5 weeks. The control mice were age and sex matched Cyp2c70 KO mice fed the same chow diet for 5 weeks. Mice were fasted for 6 hours, from 9:00 am-3:00 pm and FITC-dextran was administered as described in the Materials and Methods section. Serum FITC-dextran concentration is shown. n=5. All results are expressed as mean±SEM.

FIG. 8 shows that G-β-MCA treatment reduces total bile acid pool and biliary bile acid hydrophobicity in Cyp2c70 KO mice. Male Cyp2c70 KO mice at 8 weeks of age were fed a G-β-MCA containing chow diet for 5 weeks. The control mice were age and sex matched Cyp2c70 KO mice fed the same chow diet for 5 weeks. Mice were fasted for 6 hours, from 9:00 am-3:00 pm, and then euthanized. Total bile acid amount is shown for whole liver (Upper left panel), gallbladder (GB) (Upper right panel), small intestine (Lower panel). and total bile acid pool. n=9-11. All results are expressed as mean±SEM.

FIG. 9 shows that G-β-MCA treatment reduces total bile acid pool and biliary bile acid hydrophobicity in Cyp2c70 KO mice. Male Cyp2c70 KO mice at 8 weeks of age were fed a G-β-MCA containing chow diet for 5 weeks. The control mice were age and sex matched Cyp2c70 KO mice fed the same chow diet for 5 weeks. Mice were fasted for 6 hours, from 9:00 am-3:00 pm, and then euthanized. Total bile acid pool is shown in upper left panel. The upper right panel shows the relative abundance of individual bile acid in gallbladder (GB) bile expressed as percentage of total bile acids concentration in gallbladder bile. n=4. Percentages of specific biliary bile acids in the total pool in shown in the lower panel. All results are expressed as mean±SEM.

FIG. 10 shows effects of G-β-MCA treatment on biliary bile acid hydrophobicity and hepatic mRNA expression in Cyp2c70 KO mice. Male Cyp2c70 KO mice at 8 weeks of age were fed G-β-MCA containing chow diet for 5 weeks. The control mice were age and sex matched Cyp2c70 KO mice fed the same chow diet for 5 weeks. Mice were fasted for 6 hours, from 9:00 am-3:00 μm, and then euthanized. The upper left panel shows a gallbladder biliary bile acid hydrophobicity index calculated based on the bile acid composition shown in FIG. 9, lower panel. The relative liver expression of CYP7A1 mRNA and CYP8B1 mRNA is shown in the upper right panel and lower panel, respectively. Control set as “1.” n=11-13. All results are expressed as mean±SEM.

FIG. 11 shows the effects of G-β-MCA treatment on small intestine bile acids, and on small intestine bile acid hydrophobicity in Cyp2c70 KO mice. Male Cyp2c70 KO mice at 8 weeks of age were fed a G-β-MCA containing chow diet for 5 weeks. The control mice were age and sex matched Cyp2c70 KO mice fed the same chow diet for 5 weeks. Mice were fasted for 6 hours, from 9:00 am-3:00 μm, and then euthanized. The relative abundance of individual bile acids in the small intestine is expressed as a percentage of total small intestine bile acids (upper panel). Small intestine biliary acid hydrophobicity is reduced after treatment (lower panel). Biliary acid hydrophobicity is calculated based on the bile acid composition shown in the upper panel. All results are expressed as mean±SEM.

FIG. 12 shows other aspects of G-β-MCA treatment in Cyp2c70 KO mice. Male Cyp2c70 KO mice at 8 weeks of age were fed a G-β-MCA containing chow diet for 5 weeks. The control mice were age and sex matched Cyp2c70 KO mice fed the same chow diet for 5 weeks. Mice were fasted for 6 hours, from 9:00 am-3:00 μm, and then euthanized. The relative abundance of G-β-MCA in the small intestine is expressed as a percentage of total small intestine bile acids (upper panel). The relative abundance of individual types of bile acids in the small intestine is expressed as an absolute amount (lower panel). n=4. All results are expressed as mean±SEM.

FIG. 13 shows the effect of G-β-MCA treatment on fecal bile acid hydrophobicity in Cyp2c70 KO mice. Male Cyp2c70 KO mice at 8 weeks of age were fed G-β-MCA containing chow diet for 5 weeks. Control mice were age and sex matched Cyp2c70 KO mice fed the same chow diet for 5 weeks. Fresh feces were collected for bile acid analysis. Fecal bile acid hydrophobicity calculated based on bile acid composition is reduced as shown in the upper panel. The relative abundance of individual bile acids in fecal samples expressed as a percentage of total fecal bile acids is shown in the lower panel. n=4. All results are expressed as mean±SEM.

FIG. 14 shows that G-β-MCA treatment increases fecal bile acid excretion in Cyp2c70 KO mice. Male Cyp2c70 KO mice at 8 weeks of age were fed G-β-MCA containing chow diet for 5 weeks. Control mice are age and sex matched Cyp2c70 KO mice fed the same chow diet for 5 weeks. Fresh feces were collected for bile acid analysis. Total fecal bile acids is shown in the upper panel. n=8-10. The relative abundance of specific individual bile acids in fecal samples expressed as an absolute amount is shown in the lower panel. n=4. All results are expressed as mean±SEM.

FIG. 15 shows the effects of G-β-MCA on Ileal FGF15 mRNA expression (upper panel), Ileal SHP mRNA expression (center panel), and Ileal ASBT mRNA expression (lower panel) in Cyp2c70 KO mice. Male Cyp2c70 KO mice at 8 weeks of age were fed G-β-MCA containing chow diet for 5 weeks. Control mice are age and sex matched Cyp2c70 KO mice fed the same chow diet for 5 weeks. Fresh feces were collected for bile acid analysis. Total RNA was extracted from ˜1 cm long terminal ileum for mRNA measurement. n=10-13. All results are expressed as mean±SEM.

FIG. 16 shows that Gly-β-MCA and UDCA treatment reduce liver injury marker in female Cyp2c70 KO mice. Female WT and Cyp2c70 KO mice at 8 weeks of age were treated with Gly-β-MCA or UDCA (160 mg/kg/day) for 4 weeks. Mice were fasted for 6 h from 9 am-3 pm and euthanized. serum alanine aminotransferase (ALT). n=4-8. All results are expressed as mean±SEM. One-way ANOVA and Tukey post hoc test were used for all statistical analysis. A p<0.05 was considered statistically significant. “*”, <0.05; “***”, <0.001; “****”<0,0001.

FIG. 17 shows that Gly-β-MCA and UDCA treatment reduce ductular reaction in female Cyp2c70 KO mice. Female WT and Cyp2c70 KO mice at 8 weeks of age were treated with Gly-β-MCA or UDCA (160 mg/kg/day) for 4 weeks. Mice were fasted for 6 h from 9 am-3 pm and euthanized. Representative image of CK19 immunostaining.

FIG. 18 shows that Gly-β-MCA and UDCA treatment reduce ductular reaction in female Cyp2c70 KO mice. Female WT and Cyp2c70 KO mice at 8 weeks of age were treated with Gly-β-MCA or UDCA (160 mg/kg/day) for 4 weeks. Mice were fasted for 6 h from 9 am-3 pm and euthanized. Quantitative measurement of CL19 positive areas. All results are expressed as mean±SEM. One-way ANOVA and Tukey post hoc test were used for all statistical analysis. A p<0.05 was considered statistically significant. “*”, <0.05; “***”, <0.001; “****”<0,0001.

FIG. 19 shows that Gly-β-MCA and UDCA treatment reduce portal fibrosis in female Cyp2c70 KO mice. Female WT and Cyp2c70 KO mice at 8 weeks of age were treated with Gly-β-MCA or UDCA (160 mg/kg/day) for 4 weeks. Mice were fasted for 6 h from 9 am-3 pm and euthanized. Representative images of Sirius Res stain. Scale bar=600 μm.

FIG. 20 shows that Gly-β-MCA and UDCA treatment reduce portal fibrosis in female Cyp2c70 KO mice. Female WT and Cyp2c70 KO mice at 8 weeks of age were treated with Gly-β-MCA or UDCA (160 mg/kg/day) for 4 weeks. Mice were fasted for 6 h from 9 am-3 pm and euthanized. Sirius Red positive area per view was quantified with ImageJ. Software. All results are expressed as mean±SEM. One-way ANOVA and Tukey post hoc test were used for all statistical analysis. A p<0.05 was considered statistically significant. “*”, <0.05; “**”, <0.01; “****”<0,0001.

### DETAILED DESCRIPTION

Therapeutic reduction of hydrophobic bile acids is considered beneficial in treatments for cholestasis in human patients. The present disclosure describes the use of certain muricholic acids (MCAs) or pharmaceutically acceptable salts thereof for reducing the bile acid pool size in all forms of genetic and acquired cholestasis conditions by reducing total and hepatic bile acid pool size and decreasing the biliary bile acid and intestinal bile acid hydrophobicity index and bile acid absorption in the intestine of the subject. This leads to an increase in bile acid excretion and a decrease in the total bile acid pool size. The method of treating the cholestasis liver condition includes administering the MCA(s) to the subject. Examples of cholestasis liver conditions which may be treated include, but are not limited to, Primary biliary cholangitis, Primary sclerosing cholangitis, Biliary atresia, Progressive familial intrahepatic cholestasis 1 (PFIC 1), Progressive familial intrahepatic cholestasis 2 (PFIC 2), Progressive familial intrahepatic cholestasis 3 (PFIC 3), and Progressive familial intrahepatic cholestasis 4 (PFIC 4). Examples of MCAs that can be used in various treatment methods herein include, but are not limited to, α-MCA, β-MCA, ω-MCA, glycine-conjugated α-MCA (G-α-MCA), glycine-conjugated β-MCA (G-β-MCA), glycine-conjugated ω-MCA (G-ω-MCA), taurine-conjugated α-MCA (T-α-MCA), taurine-conjugated β-MCA (T-β-MCA), and taurine-conjugated ω-MCA (T-ω-MCA). In another embodiment, the MCAs, such as Gly-β-MCA, can be used in combination with the FGF19 analog NGM282 (also known as Aldafermin), which inhibits CYP7A1 and bile acid synthesis. In another embodiment, the MCAs, such as Gly-β-MCA, can be used in combination with GalNac siCYP7A1 (an siRNA targeted to the liver for inhibiting the bile acid synthesis rate limiting enzyme CYP7A1).

Before further describing various embodiments of the present disclosure in more detail by way of exemplary description, examples, and results, it is to be understood that the compounds, compositions, and methods of present disclosure are not limited in application to the details of specific embodiments and examples as set forth in the following description. The description provided herein is intended for purposes of illustration only and is not intended to be construed in a limiting sense. As such, the language used herein is intended to be given the broadest possible scope and meaning; and the embodiments and examples are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the present disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications and equivalents apparent to those having ordinary skill in the art are included within the scope of the present disclosure. All of the compounds, compositions, and methods and application and uses thereof disclosed herein can be made and executed without undue experimentation in light of the present disclosure. Thus, while the compounds, compositions, and methods of the present disclosure have been described in terms of particular embodiments, it will be apparent to those of skill in the art that variations may be applied to the compounds, compositions, and methods and in the steps or in the sequence of steps of the methods described herein without departing from the concept, spirit, and scope of the inventive concepts.

All patents, published patent applications, and non-patent publications including published articles mentioned in the specification or referenced in any portion of this application, are herein expressly incorporated by reference in their entirety to the same extent as if each individual patent or publication was specifically and individually indicated to be incorporated by reference.

Unless otherwise defined herein, scientific and technical terms used in connection with the present disclosure shall have the meanings that are commonly understood by those having ordinary skill in the art.

The following abbreviations may be used herein:
ALT: alanine aminotransferase;
ASBT: apical sodium-dependent bile acid transporter;
AST: aspartate aminotransferase;
CA: cholic acid;
CDCA: chenodeoxycholic acid;
CK19: cytokeratin-19;
COL1A1: collagen, type I, α1;
CYP7A1: cholesterol 7α-hydroxylase;
CYP8B1: cholesterol 12α-hydroxylase;
Da: Dalton;
DCA: deoxycholic acid;
FGF15: fibroblast growth factor 15;
FGF19: fibroblast growth factor 19;
FITC-dextran: fluorescein isothiocyanate-conjugated-dextran;
FXR: farnesoid X receptor;
G-α-MCA: glycine-conjugated α-muricholic acid;
G-β-MCA: glycine-conjugated β-MCA;
G-ω-MCA: glycine-conjugated ω-MCA;
T-α-MCA: taurine-conjugated α-MCA;
T-β-MCA: taurine-conjugated β-MCA;
T-ω-MCA: taurine-conjugated ω-MCA;
H&E: hematoxylin and eosin;
IBAT: ileal bile acid transporter;
IL1β: interleukin 1β;
IL6: interleukin 6;
KO: knockout;
LCA: lithocholic acid;
MCA: muricholic acid;
MCP-1: monocyte chemoattractant protein-1;
mRNA: messenger ribonucleic acid;
MW: molecular weight,
ND: not detectable;
OCA: obeticholic acid;
PCR: Polymerase Chain Reaction;
RNA: ribonucleic acid;
SEM: standard error of the mean;
SHP: small heterodimer partner;
T-CA: tauro-conjugated cholic acid;
T-CDCA: tauro-conjugated CDCA;
T-DCA: tauro-deoxycholic acid;
TGR5: Takeda G protein-coupled receptor 5;
TIMP1: metallopeptidase inhibitor 1;
T-LCA: tauro-lithocholic acid;
TNFα: tumor necrosis factor α;
UDCA: ursodeoxycholic acid;
WT: wild type;
wt/wt: weight-to-weight; and
ZO-1: zonula occuldens-1.

As utilized in accordance with the methods, compounds, and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. Further, unless otherwise required by context, singular terms shall include pluralities and plural terms shall include the singular. Where used herein, the specific term “single” is limited to only “one.”

Where used herein, the pronoun “we” is intended to refer to all persons involved in a particular aspect of the investigation disclosed herein and as such may include non-inventor laboratory assistants and collaborators working under the supervision of the inventor.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. For example, reference to a range of 1-50 therefore includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc., all the way down to the number one (1).

As used in this specification and claims, the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the composition, the method used to administer the active agent or composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately,” where used herein when referring to a measurable value such as an amount, a temporal duration, and the like, is meant to encompass, for example, variations of ±20%, or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment. Further, all references to one or more embodiments or examples are to be construed as non-limiting to the claims.

By “biologically active” is meant the ability of the active agent to modify the physiological system of an organism without reference to how the active agent has its physiological effects. The effectiveness of a method or use, such as a treatment that provides a potential therapeutic benefit or improvement of a condition or disease, can be ascertained by various methods and testing assays.

The active agents which comprise the combination treatments disclosed herein can be used in treatments of various cholestasis liver conditions, including but not limited to Primary biliary cholangitis, Primary sclerosing cholangitis, Biliary atresia, Progressive familial intrahepatic cholestasis 1 (PFIC 1), Progressive familial intrahepatic cholestasis 2 (PFIC 2), Progressive familial intrahepatic cholestasis 3 (PFIC 3), and Progressive familial intrahepatic cholestasis 4 (PFIC 4).

The term “pharmaceutically acceptable” refers to compounds and compositions which are suitable for administration to humans and/or animals without undue adverse side effects such as toxicity, irritation and/or allergic response commensurate with a reasonable benefit/risk ratio. The compounds or conjugates of the present disclosure may be combined with one or more pharmaceutically-acceptable excipients, including carriers, vehicles, diluents, and adjuvants which may improve solubility, deliverability, dispersion, stability, and/or conformational integrity of the compounds or conjugates thereof.

The term “active agent” as used herein refers to, in certain non-limiting embodiments, α-MCA, β-MCA, ω-MCA, G-α-MCA, G-β-MCA, G-ω-MCA, T-α-MCA, T-β-MCA, and T-ω-MCA, and pharmaceutically acceptable salts thereof. In some embodiments the active agent is conjugated to a targeting moiety to form a conjugate. A conjugate is a compound comprising an active agent covalently linked, directly or indirectly via a linker molecule, to a secondary compound, such as an antibody or fragment thereof.

The active agent may be associated with a targeting moiety or molecule which is able to bind to a target cell such as a hepatocyte or a portion of a target cell. The targeting moiety may be linked directly or indirectly to the active agent, or to the pharmaceutically acceptable carrier, vehicle, or diluent which contains or is associated with the active agent. The targeting moiety may be any molecule that can bind to another molecule. For example, a targeting moiety may include an antibody or its antigen-binding fragments, a receptor molecule, a chimeric antibody molecule, or an affinity reagent. As used herein, the term “targeting moiety” refers to a structure that binds or associates with a biological moiety or fragment thereof.

As noted, in some embodiments, the targeting moiety may be an antibody. In some embodiments, the targeting moiety may be a monoclonal antibody (mAb). In some embodiments, the targeting moiety may be an antibody fragment, surrogate, or variant. In some embodiments, the targeting moiety may be a protein ligand. In some embodiments, the targeting moiety may be a protein scaffold. In some embodiments, the targeting moiety may be a peptide. In some embodiments, the targeting moiety may be RNA or DNA. In some embodiments, the targeting moiety may be an RNA or DNA fragment. In some embodiments, the targeting moiety may be a small molecule ligand.

In at least certain compounds of the present disclosure, polyethylene glycol (PEG) molecules (also known as poly(ethylene oxide) and poly(oxyethylene)) are used, for example as linkers to link other compounds together to for drug conjugates. PEG comprises repeating units of ethylene glycol and is available in different average MWs based on the average number of ethylene glycol units in the PEG molecules of the particular PEG composition. For example, PEG₈₈, a PEG molecule with 2 ethylene glycol units, has a MW of 88 Da. PEG₄₀₀, a PEG molecule with about 8 ethylene glycol units, has a MW of 400 Da. PEG_{60,000}, a PEG molecule with about 1364 ethylene glycol units, has a MW of about 60,000 Da. The PEG molecule may comprise up to 30,000 ethylene glycol units, Other examples include, but are not limited to, PEG₂₀₀ having an average MW of about 200 Da, PEG₃₀₀ having an average MW of about 300 Da, PEG₄₀₀ having an average MW of about 400 Da, PEG₅₀₀ having an average MW of about 500 Da, PEG₇₅₀ having an average MW of about 750 Da, PEG₁₀₀₀ having an average MW of about 1000 Da, PEG₁₅₀₀ having an average MW of about 1500 Da, PEG₂₀₀₀ having an average MW of about 2000 Da, PEG₃₀₀₀ having an average MW of about 3000 Da, PEG₃₃₅₀ having an average MW of about 3350 Da, PEG₃₅₀₀ having an average MW of about 3500 Da, PEG₄₀₀₀ having an average MW of about 4000 Da, PEG₅₀₀₀ having an average MW of about 5000 Da, PEG₆₀₀₀ having an average MW of about 6000 Da, PEG₇₅₀₀ having an average MW of about 7500 Da, PEG 10,000 having an average MW of about 10,000 Da, PEG_{15,000} having an average MW of about 15,000 Da, PEG_{20,000} having an average MW of about 20,000 Da, PEG_{25,000} having an average MW of about 25,000 Da, PEG 30,000 having an average MW of about 30,000 Da, PEG_{40,000} having an average MW of about 40,000 Da, PEG_{50,000} having an average MW of about 50,000 Da, and PEG_{60,000} having an average MW of about 60,000 Da. Where used herein the term PEG is intended to refer to any of the examples of PEG listed above, and to PEGs having MWs in the range between 88 Da and 60,000 Da, unless a particular MW is specified. In other embodiments, the linker molecule may be an amino acid, a peptide, or a polypeptide.

In various non-limiting embodiments, the drug conjugates of the present disclosure include active agents which are linked via a linker (e.g., a PEG, amino acid, peptide or polypeptide) to an anchor-solubilizing moiety such as a phosphatidylethanolamine (PE). Examples of such PEs include but are not limited to distcaroylphosphatidylethanolamine (DSPE), dipalmitoylphosphatidylethanolamine, dimyristoylphosphatidylethanolamine, diarachidylphosphatidylethanolamine, diolcylphosphatidylethanolamine, myristoylstearoylphosphatidylethanolamine, laurylstcaroylphosphatidylethanolamine, myristoylpalmitoylphosphatidylethanolamine, laurylpalmitoylphosphatidylethanolamine, laurylmyristoylphosphatidylethanolamine, olcylpalmitoylphosphatidylethanolamine, dilaurylphosphatidylethanolamine, palmitoylstearoylphosphatidylethanolamine, arachidylstearoylphosphatidylethanolamine, olcylstearoylphosphatidylethanolamine, arachidylpalmitoylphosphatidylethanolamine, arachidylmyristoylphosphatidylethanolamine, laurylarachidylphosphatidylethanolamine, olcylmyristoylphosphatidylethanolamine, oleylarachidylphosphatidylethanolamine, and lauryloleylphosphatidylethanolamine. In other embodiments, anchoring/solubilizing moiety may comprise any one of the above moieties wherein the ethanolamine is substituted with serine (forming a phosphatidylserine (PS)) or choline (forming a phosphatidylcholine (PC)), such as distearoylphosphatidylserine or distearoylphosphatidylcholine. In other embodiments, the anchoring/solubilizing moiety may comprise a combination of two or more of the above moieties. In other embodiments, anchoring/solubilizing moiety may comprise a single saturated, unsaturated, or polyunsaturated lipid molecule comprising 2-28 carbon atoms, particularly 10-18 carbon atoms, such as a saturated, unsaturated, or polyunsaturated fatty acid. The anchor-solubilizing moiety may comprise a PE, PS or PC with a single fatty acid or two fatty acids, which may be selected from the group of saturated, unsaturated, and polyunsaturated fatty acids.

In particular, non-limiting examples, the targeting vector of the present disclosure is combined with liposomes in which a cargo molecule is disposed. In addition to other pharmaceutically acceptable carrier(s), the liposome may contain amphipathic agents such as lipids which exist in an aggregated form as micelles, insoluble monolayers, liquid crystals, or lamellar layers in aqueous solution. Suitable lipids for liposomal formulation include, but are not limited to, monoglycerides, diglycerides, sulfatides, lysolecithin, phospholipids, saponin, bile acids, combinations thereof, and the like. Preparation of such liposomal formulations is well within the level of ordinary skill in the art, as disclosed, for example, in U.S. Pat. Nos. 4,235,871; 4,501,728; 4,837,028; and 4,737,323; the entire contents of each of which are incorporated herein by reference. As used herein, the term “liposome” means a vesicle composed of amphiphilic lipids arranged in a spherical bilayer or bilayers. Liposomes are unilamellar or multilamellar vesicles which have a membrane formed from a lipophilic material and an aqueous interior. The aqueous portion contains the active agent to be delivered. Liposomes can be made from phospholipids other than naturally-derived phosphatidylcholine. Neutral liposome compositions, for example, can be formed from dimyristoyl phosphatidylcholine (DMPC) or dipalmitoyl phosphatidylcholine (DPPC) or other similar lipids. Anionic liposome compositions generally are formed from dimyristoyl phosphatidylglycerol, while anionic fusogenic liposomes are formed primarily from diolcoyl phosphatidylethanolamine (DOPE). Another type of liposomal composition is formed from phosphatidylcholine (PC) such as, for example (but not by way of limitation), soybean PC, and egg PC. Another type is formed from mixtures of phospholipid and/or phosphatidylcholine and/or cholesterol.

The term “antibody” as used herein can refer to both intact “full length” antibodies as well as to antigen-binding fragments thereof (unless otherwise explicitly noted). The afore-mentioned antigen-binding fragments may also be referred to herein as antigen binding fragments, antigen binding compounds, antigen binding portions, binding fragments, binding portions, or antibody fragments. Also, as used herein, the term “antibody” includes, but is not limited to, synthetic antibodies, monoclonal antibodies, recombinantly produced antibodies, intrabodies, multispecific antibodies (including bi-specific antibodies), human antibodies, humanized antibodies, chimeric antibodies, recombinant single chain polypeptide molecules in which light and heavy chain variable regions are connected by a peptide linker, i.e., single-chain Fv (scFv) fragments, bivalent scFv (bi-scFv), trivalent scFv (tri-scFv), Fab fragments, Fab′ fragments, F(ab′) fragments, F(ab′)₂ fragments, F(ab)₂ fragments, disulfide-linked Fvs (sdFv) (including bi-specific sdFvs), and anti-idiotypic (anti-Id) antibodies, dAb fragments, nanobodies, diabodies, triabodies, tetrabodies, linear antibodies, isolated CDRs, and epitope-binding fragments of any of the above. Regardless of structure, an antibody fragment refers to an isolated portion of the antibody that binds to the same antigen that is recognized by the intact antibody.

The antibodies of several embodiments provided herein may be monospecific, bispecific, trispecific, or of greater multispecificity, such as multispecific antibodies formed from antibody fragments. The term “antibody” also includes a diabody (homodimeric Fv fragment) or a minibody (V_{L}-V_{H}—C_{H3}), a bispecific antibody, or the like. A bispecific or bifunctional antibody is an artificial hybrid antibody having two different heavy/light chain pairs and two different binding sites. Multispecific antibodies may be specific for different epitopes of a polypeptide or may be specific for both a polypeptide as well as for a heterologous epitope, such as a heterologous polypeptide or solid support material. Single chain antibodies produced by joining antibody fragments using recombinant methods, or a synthetic linker, are also encompassed by the present disclosure (e.g., see, for example, International Patent Application Publication Nos. WO 93/17715; WO 92/08802; WO 91/00360; and WO 92/05793; and U.S. Pat. Nos. 4,474,893; 4,714,681; 4,925,648; 5,573,920; and 5,601,819).

In at least certain embodiments of the present disclosure, the term “therapeutic agent” refers to an active agent comprising an antibody and/or antibody-derived compound or another compound as described herein.

A “diagnostic agent,” which may also be referred to herein as an “imaging agent,” is a substance that is useful in diagnosing a disease or imaging a cell or tissue. Useful diagnostic agents of the present disclosure may include antibodies and antibody-derived compounds described herein and may further comprise by linkage or other association radioisotopes, dyes, contrast agents, fluorescent compounds or molecules, and enhancing agents (e.g., paramagnetic ions).

As used herein, “pure” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

Non-limiting examples of animals within the scope and meaning of this term include dogs, cats, rats, mice, guinea pigs, chinchillas, horses, goats, cattle, sheep, zoo animals, Old and New World monkeys, non-human primates, and humans.

“Treatment” refers to therapeutic treatments. “Prevention” refers to prophylactic or preventative treatment measures or reducing the onset of a condition or disease. The term “treating” refers to administering the active agent to a subject for therapeutic purposes and/or for prevention. Non-limiting examples of modes of administration include oral, topical, retrobulbar, subconjunctival, transdermal, parenteral, subcutaneous, intranasal, intramuscular, intraperitoneal, intravitreal, and intravenous routes, including both local and systemic applications. In addition, the active agent of the present disclosure may be designed to provide delayed, controlled, extended, and/or sustained release using formulation techniques which are well known in the art.

The term “topical” is used herein to define a mode of administration through an internal or external epithelial surface, such as but not limited to, a material that is administered by being applied externally to the eye or a nasal mucosa. A non-limiting example of topical administration is the use of eyedrops or the use of a nasally-administered aerosol.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

The term “effective amount” refers to an amount of the active agent which is sufficient to exhibit a detectable therapeutic or treatment effect in a subject without excessive adverse side effects (such as substantial toxicity, irritation and allergic response) commensurate with a reasonable benefit/risk ratio when used in the manner of the present disclosure. The effective amount for a subject will depend upon the subject's type, size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

The term “ameliorate” means a detectable or measurable improvement in a subject's condition or symptom thereof. A detectable or measurable improvement includes a subjective or objective decrease, reduction, inhibition, suppression, limit or control in the occurrence, frequency, severity, progression, or duration of the condition, or an improvement in a symptom or an underlying cause or a consequence of the condition, or a reversal of the condition. A successful treatment outcome can lead to a “therapeutic effect,” or “benefit” of ameliorating, decreasing, reducing, inhibiting, suppressing, limiting, controlling or preventing the occurrence, frequency, severity, progression, or duration of a condition, or consequences of the condition in a subject.

A decrease or reduction in worsening, such as stabilizing the condition, is also a successful treatment outcome. A therapeutic benefit therefore need not be complete ablation or reversal of the condition, or any one, most or all adverse symptoms, complications, consequences or underlying causes associated with the condition. Thus, a satisfactory endpoint may be achieved when there is an incremental improvement such as a partial decrease, reduction, inhibition, suppression, limit, control or prevention in the occurrence, frequency, severity, progression, or duration, or inhibition or reversal of the condition (e.g., stabilizing), over a short or long duration of time (e.g., seconds, minutes, hours).

The active agents of the present disclosure may be present in the pharmaceutical compositions at any concentration that allows the pharmaceutical composition to function in accordance with the present disclosure; for example, but not by way of limitation, the active agents may be present in the composition in a percent wt/wt range having a lower level selected from 0,0001%, 0.005%, 0.001%, 0.005%, 0.01%, 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9% and 2.0%; and an upper level selected from 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, and 95%. Non-limiting examples of particular percent wt/wt ranges include a range of from about 0,0001% to about 95%, a range of from about 0.001% to about 75%; a range of from about 0.005% to about 50%; a range of from about 0.01% to about 40%; a range of from about 0.05% to about 35%; a range of from about 0.1% to about 30%; a range of from about 0.1% to about 25%; a range of from about 0.1% to about 20%; a range of from about 1% to about 15%; a range of from about 2% to about 12%; a range of from about 5% to about 10%; and the like. Any other range that includes a lower level selected from the above-listed lower-level concentrations and an upper level selected from the above-listed upper-level concentrations also falls within the scope of the present disclosure.

Suitable carriers, vehicles, and other components that may be included in the formulation are described, for example, in Remington: The Science and Practice of Pharmacy, 21^{st} Ed. and 22^{nd} Ed. The term “pharmaceutically acceptable” means that the carrier is a non-toxic material that does not interfere with the effectiveness of the biological activity of the active agent. The characteristics of the carrier will depend on various factors, including but not limited to, the route of administration.

For example, but not by way of limitation, the active agent may be dissolved in a physiologically acceptable pharmaceutical carrier or diluent and administered as either a solution or a suspension. Non-limiting examples of suitable pharmaceutically acceptable carriers include water, saline, dextrose solutions, fructose solutions, ethanol, or oils of animal, vegetative, or synthetic origin, or any combination thereof. A sterile diluent, which may contain materials generally recognized for approximating physiological conditions and/or as required by governmental regulations, may be employed as the pharmaceutically acceptable carrier. In this respect, the sterile diluent may contain a buffering agent to obtain a physiologically acceptable pH, such as (but not limited to) sodium chloride, saline, phosphate-buffered saline, and/or other substances which are physiologically acceptable and/or safe for use.

The pharmaceutical compositions may also contain one or more additional components in addition to the active agent and pharmaceutically acceptable carrier(s) (and other additional therapeutically active agent(s) if present). Examples of additional components that may be present include, but are not limited to, diluents, fillers, salts, buffers, preservatives, stabilizers, solubilizers, and other materials well known in the art. Another non-limiting example of an additional component that may be present in the pharmaceutical composition is a delivery agent, as discussed in further detail herein below.

Other embodiments of the pharmaceutical compositions of the present disclosure may include the incorporation or entrapment of the active agent in various types of drug delivery systems that function to provide targeted delivery, controlled release, and/or increased half-life to the active agent. For example, but not by way of limitation, it is possible to entrap the active agent in microcapsules prepared by coacervation techniques or by interfacial polymerization (for example, hydroxymethylcellulose or gelatin-microcapsules and poly-(methylmethacylate) microcapsules, respectively). It is also possible to entrap the active agent in macroemulsions or colloidal drug delivery systems (such as but not limited to, liposomes, albumin microspheres, microemulsions, nanoparticles, nanocapsules, and the like). Such techniques are well known to persons having ordinary skill in the art, and thus no further description thereof is deemed necessary.

In one particular, non-limiting example, the pharmaceutical composition may include a liposome in which the active agent is disposed. In addition to other pharmaceutically acceptable carrier(s), the liposome may contain amphipathic agents such as lipids which exist in an aggregated form as micelles, insoluble monolayers, liquid crystals, or lamellar layers in aqueous solution. Suitable lipids for liposomal formulation include, but are not limited to, monoglycerides, diglycerides, sulfatides, lysolecithin, phospholipids, saponin, bile acids, combinations thereof, and the like. Preparation of such liposomal formulations is well within the level of ordinary skill in the art, as disclosed, for example, in U.S. Pat. Nos. 4,235,871; 4,501,728; 4,837,028; and 4,737,323; the entire contents of each of which are incorporated herein by reference.

In other non-limiting examples, the active agent of the present disclosure may be incorporated into particles of one or more polymeric materials, as this type of incorporation can be useful in controlling the duration of action of the active agent by allowing for controlled release from the preparations, thus increasing the half-life thereof. Non-limiting examples of polymeric materials that may be utilized in this manner include polyesters, polyamides, polyamino acids, hydrogels, poly(lactic acid), ethylene vinylacetate copolymers, copolymer micelles of, for example, PEG and poly(l-aspartamide), and combinations thereof.

The pharmaceutical compositions described or otherwise contemplated herein may further comprise at least one delivery agent, such as a targeting moiety, that assists in delivery of the active agent to a desired site of delivery, such as a liver hepatocyte.

The compositions of the present disclosure may be formulated for administration by any other method known or otherwise contemplated in the art, as long as the route of administration allows for delivery of the active agent so that the compounds can function in accordance with the present disclosure, e.g., to reduce cholestasis. Examples of other routes of administration include, but are not limited to, oral, topical, retrobulbar, subconjunctival, transdermal, parenteral, subcutaneous, intranasal, intramuscular, intraperitoneal, intravitreal, and intravenous routes, including both local and systemic application routes.

Another non-limiting embodiment of the present disclosure is directed to a kit that contain one or more of any of the pharmaceutical compositions described or otherwise contemplated herein. The kit may further contain a second agent as described herein above for use concurrently with the pharmaceutical composition(s). If the composition present in the kit is not provided in the form in which it is to be delivered, the kit may further contain a pharmaceutically acceptable carrier, vehicle, diluent, or other agent for mixing with the active agent for preparation of the pharmaceutical composition. The kit including the composition and/or other reagents may also be packaged with instructions packaged for administration and/or dosing of the compositions contained in the kit. The instructions may be fixed in any tangible medium, such as printed paper, or a computer-readable magnetic or optical medium, or instructions to reference a remote computer data source such as a worldwide web page accessible via the internet.

The kit may contain single or multiple doses of the pharmaceutical composition which contains the active agent. When multiple doses are present, the doses may be disposed in bulk within a single container, or the multiple doses may be disposed individually within the kit; that is, the pharmaceutical compositions may be present in the kit in unit dosage forms to facilitate accurate dosing. The term “unit dosage forms” as used herein refers to physically discrete units suitable as unitary dosages for human subjects and other mammals; each unit contains a predetermined quantity of the active agent calculated to produce the desired therapeutic effect, in association with a suitable pharmaceutical excipient. Typical unit dosage forms of liquid compositions include prefilled, premeasured ampules or syringes; for solid compositions, typical unit dosage forms include pills, tablets, capsules, or the like. In such compositions, the active agent may sometimes be a minor component (from about 0.1 to about 50% by weight, such as but not limited to, from about 1 to about 40% by weight) with the remainder being various vehicles or carriers and processing aids helpful for forming the desired dosing form.

The active agent may be provided as a “pharmaceutically acceptable salt,” which refers to salts that retain the biological effectiveness and properties of a compound and, which are not biologically or otherwise undesirable for use in a pharmaceutical. In many cases, the compounds disclosed herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred (but not by way of limitation) are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297 (incorporated by reference herein in its entirety).

The amount of the active agent that is effective in the treatment described herein can be determined by the attending diagnostician, as one of ordinary skill in the art, by the use of conventional techniques and by observing results obtained under analogous circumstances. In determining the therapeutically effective dose, a number of factors may be considered by the attending diagnostician, including, but not limited to: the species of the subject; its size, age, and general health; the specific diseases or other conditions involved; the degree, involvement, and/or severity of the diseases or conditions; the response of the individual subject; the particular active agent administered; the mode of administration; the bioavailability characteristics of the preparation administered; the dose regimen selected; the use of concomitant medication; and other relevant circumstances. A therapeutically effective amount of an active agent of the present disclosure also refers to an amount of the active agent which is effective in controlling, reducing, or ameliorating the condition to be treated.

Practice of the method of the present disclosure may include administering to a subject a therapeutically effective amount of the pharmaceutical composition (containing the active agent in any suitable systemic and/or local formulation, in an amount effective to deliver the dosages listed above. The dosage can be administered, for example, but not by way of limitation, on a one-time basis, or administered at multiple times (for example, but not by way of limitation, from one to five times per day, or once or twice per week). The pharmaceutical composition may be administered either alone or in combination with other therapies, in accordance with the inventive concepts disclosed herein.

Compositions of the active agent can be administered in a single dose treatment or in multiple dose treatments on a schedule and over a time period appropriate to the age, weight and condition of the subject, the particular composition used, and the route of administration. In one embodiment, a single dose of the composition according to the disclosure is administered. In other embodiments, multiple doses are administered. The frequency of administration can vary depending on any of a variety of factors, e.g., severity of the symptoms, or whether the composition is used for prophylactic or curative purposes. For example, in certain embodiments, the composition is administered once per month, twice per month, three times per month, every other week, once per week, twice per week, three times per week, four times per week, five times per week, six times per week, every other day, daily, twice a day, or three times a day. The duration of treatment, e.g., the period of time over which the composition is administered, can vary, depending on any of a variety of factors, e.g., subject response. For example, the composition can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

The compositions can be combined with a pharmaceutically acceptable carrier (excipient) or vehicle to form a pharmacological composition. Pharmaceutically acceptable carriers can contain a physiologically acceptable compound that acts to, e.g., stabilize, or increase or decrease the absorption or clearance rates of the pharmaceutical compositions. Physiologically acceptable carriers and vehicles can include, for example, carbohydrates, such as glucose, sucrose, or dextrans, antioxidants, such as ascorbic acid or glutathione, chelating agents, low molecular weight proteins, detergents, liposomal carriers, or excipients or other stabilizers and/or buffers. Other physiologically acceptable compounds, carriers, and vehicles include wetting agents, emulsifying agents, dispersing agents or preservatives.

When administered orally, the present compositions may be protected from digestion. This can be accomplished either by complexing the active agent with a composition to render it resistant to acidic and enzymatic hydrolysis or by packaging active agent in an appropriately resistant carrier such as a liposome, e.g., such as shown in U.S. Pat. No. 5,391,377.

For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. For topical, transdermal administration, the agents are formulated into ointments, creams, salves, powders and gels. Transdermal delivery systems can also include, e.g., patches. The present compositions can also be administered in sustained delivery or sustained release mechanisms. For example, biodegradable microspheres or capsules or other biodegradable polymer configurations capable of sustained delivery of the active agent can be included herein.

For inhalation, the active agent can be delivered using any system known in the art, including dry powder aerosols, liquids delivery systems, air jet nebulizers, propellant systems, and the like. For example, the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include, e.g., air jet nebulizers.

The active agent can be delivered alone or as pharmaceutical compositions by any means known in the art, e.g., systemically, regionally, or locally; by intra-arterial, intrathecal (IT), intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa).

In one aspect, the pharmaceutical formulations comprising the active agent are incorporated in lipid monolayers or bilayers, e.g., liposomes, such as shown in U.S. Pat. Nos. 6,110,490; 6,096,716; 5,283,185; and 5,279,833. Liposomes and liposomal formulations can be prepared according to standard methods and are also well known in the art, such as U.S. Pat. Nos. 4,235,871; 4,501,728 and 4,837,028.

In one aspect, the active agent is prepared with one or more carriers that will protect the active agent against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

The active agent in general may be formulated to obtain compositions that include one or more pharmaceutically suitable excipients, surfactants, polyols, buffers, salts, amino acids, or additional ingredients, or some combination of these. This can be accomplished by known methods to prepare pharmaceutically useful dosages, whereby the active agent is combined in a mixture with one or more pharmaceutically suitable excipients. Sterile phosphate-buffered saline is one example of a pharmaceutically suitable excipient.

Non-limiting examples of routes of administration of the active agents described herein include parenteral injection, e.g., by subcutaneous, intramuscular or transdermal delivery. Other forms of parenteral administration include intravenous, intraarterial, intralymphatic, intrathecal, intraocular, intracerebral, or intracavitary injection. In parenteral administration, the compositions will be formulated in a unit dosage injectable form such as a solution, suspension or emulsion, in association with a pharmaceutically acceptable excipient. Such excipients are inherently nontoxic and nontherapeutic. Non-limiting examples of such excipients are saline, Ringer's solution, dextrose solution, and Hanks' solution. Nonaqueous excipients such as fixed oils and ethyl oleate may also be used. An alternative excipient is 5% dextrose in saline. The excipient may contain minor amounts of additives such as substances that enhance isotonicity and chemical stability, including buffers and preservatives.

Formulated compositions comprising the active agent can be used for subcutaneous, intramuscular or transdermal administration. Compositions can be presented in unit dosage form, e.g., in ampoules or in multi-dose containers, with an added preservative. Compositions can also take such forms as suspensions, solutions or emulsions in oily or aqueous vehicles, and can contain formulatory agents such as suspending, stabilizing, and/or dispersing agents. Alternatively, the compositions can be in powder form for constitution with a suitable vehicle, e.g., sterile pyrogen-free water, before use.

The active agents may be administered in solution. The formulation thereof may be in a solution having a suitable pharmaceutically acceptable buffer such as phosphate, Tris (hydroxymethyl) aminomethane-HCl or citrate, and the like. Buffer concentrations should be in the range of 1 to 100 mM. The formulated solution may also contain a salt, such as sodium chloride or potassium chloride in a concentration of 50 to 150 mM. An effective amount of a stabilizing agent such as mannitol, trehalose, sorbitol, glycerol, albumin, a globulin, a detergent, a gelatin, a protamine or a salt of protamine may also be included.

For example, but not by way of limitation, the therapeutically effective amount of an active agent used in the present disclosure will generally contain sufficient active agent to deliver in a range of from about 0.01 μg/kg to about 250 mg/kg (weight of active agent/body weight of patient). For example, but not by way of limitation, the composition will deliver about 10 μg/kg to about 250 mg/kg, and more particularly about 50 μg/kg to about 200 mg/kg.

Particular ranges for a therapeutically or prophylactically effective amount of a fixed daily dose of the active agent include but are not limited to 0.01 mg/kg of the subject's body weight to 1500 mg/kg of the subject's body weight, more typically 0.01 mg/kg to 500 mg/kg, 0.1 mg/kg to 500 mg/kg, 0.1 mg/kg to 250 mg/kg, 1 mg/kg to 300 mg/kg, or 1 mg/kg to 200 mg/kg, or 1 mg/kg to 100 mg/kg, 2 mg/kg to 250 mg/kg, 3 mg/kg to 300 mg/kg, 4 mg/kg to 240 mg/kg, or 5 mg/kg to 500 mg/kg, or 5 mg/kg to 250 mg/kg, 5 mg/kg to 200 mg/kg, 7.5 mg/kg to 500 mg/kg, 7.5 mg/kg to 250 mg/kg, or 10 mg/kg to 1000 mg/kg, or 10 mg to 750 mg, 10 mg/kg to 500 mg/kg, 10 mg/kg to 400 mg/kg, or 10 mg/kg to 300 mg/kg, or 10 mg/kg to 250 mg/kg, or 10 mg/kg to 200 mg/kg, or 20 mg/kg to 1000 mg/kg, or 20 mg to 750 mg, 20 mg/kg to 500 mg/kg, 20 mg/kg to 400 mg/kg, or 20 mg/kg to 300 mg/kg, or 20 mg/kg to 250 mg/kg, or 20 mg/kg to 200 mg/kg, or 30 mg/kg to 1000 mg/kg, or 30 mg to 750 mg, 30 mg/kg to 500 mg/kg, 30 mg/kg to 400 mg/kg, or 30 mg/kg to 300 mg/kg, or 30 mg/kg to 250 mg/kg, or 30 mg/kg to 200 mg/kg, or 40 mg/kg to 1000 mg/kg, or 40 mg to 750 mg, 40 mg/kg to 500 mg/kg, 40 mg/kg to 400 mg/kg, or 40 mg/kg to 300 mg/kg, or 40 mg/kg to 250 mg/kg, or 40 mg/kg to 200 mg/kg, or 50 mg/kg to 1000 mg/kg, or 50 mg to 750 mg, 50 mg/kg to 500 mg/kg, 50 mg/kg to 400 mg/kg, or 50 mg/kg to 300 mg/kg, or 50 mg/kg to 250 mg/kg, or 50 mg/kg to 200 mg/kg, or 60 mg/kg to 1000 mg/kg, or 60 mg to 750 mg, 60 mg/kg to 500 mg/kg, 60 mg/kg to 400 mg/kg, or 60 mg/kg to 300 mg/kg, or 60 mg/kg to 250 mg/kg, or 60 mg/kg to 200 mg/kg, or 70 mg/kg to 1000 mg/kg, or 70 mg to 750 mg, 70 mg/kg to 500 mg/kg, 70 mg/kg to 400 mg/kg, or 70 mg/kg to 300 mg/kg, or 70 mg/kg to 250 mg/kg, or 70 mg/kg to 200 mg/kg, or 80 mg/kg to 1000 mg/kg, or 80 mg to 750 mg, 80 mg/kg to 500 mg/kg, 80 mg/kg to 400 mg/kg, or 80 mg/kg to 300 mg/kg, or 80 mg/kg to 250 mg/kg, or 80 mg/kg to 200 mg/kg, or 90 mg/kg to 1000 mg/kg, or 90 mg to 750 mg, 90 mg/kg to 500 mg/kg, 90 mg/kg to 400 mg/kg, or 90 mg/kg to 300 mg/kg, or 90 mg/kg to 250 mg/kg, or 90 mg/kg to 200 mg/kg, or 100 mg/kg to 1000 mg/kg, or 100 mg to 750 mg, 100 mg/kg to 500 mg/kg, 100 mg/kg to 400 mg/kg, or 100 mg/kg to 300 mg/kg, or 100 mg/kg to 250 mg/kg, or 100 mg/kg to 200 mg/kg, or 110 mg/kg to 1000 mg/kg, or 110 mg to 750 mg, 110 mg/kg to 500 mg/kg, 110 mg/kg to 400 mg/kg, or 110 mg/kg to 300 mg/kg, or 110 mg/kg to 250 mg/kg, or 110 mg/kg to 200 mg/kg, or 120 mg/kg to 1000 mg/kg, or 120 mg to 750 mg, 120 mg/kg to 500 mg/kg, 120 mg/kg to 400 mg/kg, or 120 mg/kg to 300 mg/kg, or 120 mg/kg to 250 mg/kg, or 120 mg/kg to 200 mg/kg, or 130 mg/kg to 1000 mg/kg, or 130 mg to 750 mg, 130 mg/kg to 500 mg/kg, 130 mg/kg to 400 mg/kg, or 130 mg/kg to 300 mg/kg, or 130 mg/kg to 250 mg/kg, or 130 mg/kg to 200 mg/kg, or 140 mg/kg to 1000 mg/kg, or 140 mg to 750 mg, 140 mg/kg to 500 mg/kg, 140 mg/kg to 400 mg/kg, or 140 mg/kg to 300 mg/kg, or 140 mg/kg to 250 mg/kg, or 140 mg/kg to 200 mg/kg, or 150 mg/kg to 1000 mg/kg, or 150 mg to 750 mg, 150 mg/kg to 500 mg/kg, 150 mg/kg to 400 mg/kg, or 150 mg/kg to 300 mg/kg, or 150 mg/kg to 250 mg/kg, or 150 mg/kg to 200 mg/kg.

The composition is formulated to contain an effective amount of the active agent, wherein the amount depends on the animal to be treated and the condition to be treated. In certain embodiments, the active agent is administered at a dose ranging from about 0.001 mg to about 10 g, from about 0.01 mg to about 10 g, from about 0.1 mg to about 10 g, from about 1 mg to about 10 g, from about 1 mg to about 9 g, from about 1 mg to about 8 g, from about 1 mg to about 7 g, from about 1 mg to about 6 g, from about 1 mg to about 5 g, from about 10 mg to about 10 g, from about 50 mg to about 5 g, from about 50 mg to about 5 g, from about 50 mg to about 2 g, from about 0.05 μg to about 1.5 mg, from about 10 μg to about 1 mg protein, from about 30 μg to about 500 μg, from about 40 μg to about 300 μg, from about 0.1 μg to about 200 mg, from about 0.1 μg to about 5 μg, from about 5 μg to about 10 μg, from about 10 μg to about 25 μg, from about 25 μg to about 50 μg, from about 50 μg to about 100 μg, from about 100 μg to about 500 μg, from about 500 μg to about 1 mg, from about 1 mg to about 2 mg. The specific dose level for any particular subject depends upon a variety of factors including the activity of the specific peptide, the age, body weight, general health, sex, diet, time of administration, route of administration, and rate of excretion, drug combination and the severity of the particular disease undergoing therapy.

The dosage of an administered active agent for humans will vary depending upon such factors as the patient's age, weight, height, sex, general medical condition and previous medical history. In certain non-limiting embodiments, the recipient is provided with a dosage of the active agent that is in the range of from about 1 mg to 1000 mg as a single dosage or infusion or single or multiple injections, although a lower or higher dosage also may be administered. The dosage may be in the range of from about 25 mg to 100 mg of the active agent per square meter (m²) of body surface area for a typical adult, although a lower or higher dosage also may be administered. Examples of dosages that may be administered to a human subject further include, for example, 1 to 500 mg, 1 to 70 mg, or 1 to 20 mg, although higher or lower doses may be used. Dosages may be repeated as needed, for example, once per week for 4-10 weeks, or once per week for 8 weeks, or once per week for 4 weeks. It may also be given less frequently, such as every other week for several months, or more frequently, such as twice weekly or by continuous infusion.

CYP2C70, which is encoded by the Cyp2c70 gene, mediates the synthesis of muricholic acids (MCAs) primarily from chenodeoxycholic acid (CDCA) in mice and is responsible for the species difference of primary bile acids in humans and mice. In comparison to human bile acid pool, the significantly less hydrophobic bile acid pool due to the presence of MCAs in mice has been a limitation of investigating bile acid-induced hepatobiliary toxicity and the pathological impact of altered bile acid composition observed in WT mice cannot be extrapolated to humans. However, the bile of Cyp2c70 knockout (KO) mice contains primarily hydrophobic CDCA and cholic acid (CA), thus, these mice exhibit a more human-like hydrophobic bile acid pool-induced hepatobiliary injury phenotype. In the present work, the therapeutic benefits of G-β-MCA in treating cholestasis were investigated. Results, as demonstrated below, indicate that in Cyp2c70 KO mice G-β-MCA treatment attenuates ductular reaction and liver fibrosis, improves gut barrier function, reduces the total bile acid pool size and biliary bile acid hydrophobicity, promotes fecal bile acid excretion, and reduces gut exposure to hydrophobic acid.

Examples of types of acquired or genetic cholestasis liver diseases or cholestasis-related liver diseases that can be treated using G-β-MCA or other active agents disclosed herein include, but are not limited to, (1) Primary biliary cholangitis, (2) primary sclerosing cholangitis, (3) biliary atresia, (4) Progressive familial intrahepatic cholestasis 1 (PFIC 1), (5) Progressive familial intrahepatic cholestasis 2 (PFIC 2), (6) Progressive familial intrahepatic cholestasis 3 (PFIC 3), and (7) Progressive familial intrahepatic cholestasis 4 (PFIC 4).

### EXPERIMENTAL

Examples are provided hereinbelow. However, the present disclosure is to be understood as not limited in its application to the specific experimentation, results, and laboratory procedures disclosed below or elsewhere herein. Rather, each example is provided as one of various embodiments and are meant to be exemplary, not exhaustive.

### Methods

### Reagents

Aspartate aminotransferase (AST) and alanine aminotransferase (ALT) assay kits were purchased from Pointe Scientific (Canton. MI). Bile acid assay kit was purchased from Diazyme Laboratories (Poway, CA). Glycine-conjugated β muricholic acid (G-β-MCA) was purchased from MedChemExpress Inc. (Monmouth Junction, NJ). F4/80 antibody (Cat #. 70076) was purchased from Cell Signaling Technology (Danvers, MA). CK19 antibody (ab52625) was purchased from Abcam (Waltham, MA). ZO-1 antibody (PA5-28858) was purchased from ThermoFisher Scientific (Grand Island, NY). Fluorescein isothiocyanate-dextran (FITC-dextran, MW: 3000-5000) was purchased from Sigma Aldrich (St. Louis, MO).

### Mice

The Cyp2c70 KO mice were generated by CRISPR/Cas-mediated genome engineering by Cyagen Biosciences Inc. (Santa Clara, CA) as described previously. Mice were housed in micro-isolator cages with Biofresh performance bedding (Pelleted cellulose) under 7:00 am-7:00 pm light cycle and 7:00 pm-7:00 am dark cycle. The standard chow diet was PicoLab Rodent Diet 20 (#5053, LabDiet, St. Louis, MO), which contained ˜13% fat calories and ˜62% carbohydrate calories. G-β-MCA was mixed with powered chow diet, which was subsequently mixed with κ% sterile water and pressed into small pellets. An estimated 20 mg/kg/day G-β-MCA intake was achieved based on 5 g/day food intake by a 25 g mouse. Mice were euthanized by isoflurane after a 6 hour fast from 9:00 am-3:00 μm and tissue and blood samples were collected. To measure gut permeability, mice were first fasted from 9:00 am-3:00 pm. Blood was collected between 2:30 pm-3:00 pm via tail snip for baseline measurement. FITC-dextran was dissolved in sterile PBS (60 mg/ml), and a single dose (600 mg/kg BW) was given via oral gavage at ˜3:00 pm. Blood was collected again at 1 hour after FITC-dextran administration. FITC fluorescence in serum samples was measured with a Tecan M200 PRO plate reader (excitation: 485 nm; emission: 530 nm). The fluorescent values measured in samples collected before FITC-dextran gavage was used as background measurement and the serum FITC-dextran concentration was calculated based on a standard curve.

### Bile Acid Analysis—Total Bile Acid Measurement and LC-MS Method

Bile acids were extracted with 90% ethanol from liver, whole gallbladder, whole small intestine with content, and dried feces as previously described. Fresh fecal samples were collected by placing an individual mouse in a jar briefly. Fecal samples were collected at around 9 am on the day of tissue collection and air dried and weighed. The fecal samples were then homogenized in 90% ethanol to extract bile acids. Bile acid pool was calculated as the sum of bile acids in liver, gallbladder and small intestine. For LC-MS measurement of bile acids, bile acid extracts were dried and resuspended in injection buffer and detected on a Thermo Scientific UltiMate 3000 UHPLC with a Waters Cortecs C18 column (Waters Acquity UPLC HSS T3 1.8 μm, 2.1×150 mm, part No. 186003540) and a TSQ Quantis triple quadrupole mass spectrometer.

### Histology and Immunohistochemistry

Paraffin embedded tissues were sectioned at 5 mm thickness and stained with Hematoxylin and Eosin (H&E) or Sirius Red (Direct Red 80 solution, Sigma #365548, St. Louis, MO). For immunohistochemistry, paraffin embedded tissue sections were deparaffinized and rehydrated, and after antigen retrieval, were blocked in PBS buffer containing 5% BSA and 5% goat serum for 1 hour. These tissue sections were incubated in primary antibodies overnight at 4° C., washed with PBS, and incubated with secondary antibodies in SignalStain® Boost IHC Detection Reagent (Cell Signaling, #8114, Danvers, MA) for 1 hour. Signal was visualized with a DAB kit (Cell Signaling, #11724, Danvers, MA). The tissue sections were counterstained with Hematoxylin. Images are acquired with an EVOS M5000 imaging system (ThermoFisher Scientific, Grand Island, NY). Images taken with 10× lens were shown as representative images. Images taken with 4× lens were used for quantification of positive stain area with ImageJ software (NIH).

### Real-time PCR

Liver total RNA was purified with Trizol (Sigma-Aldrich, St. Louis, MO). Total RNA (2 mg) was used in reverse transcription reaction with Oligo dT primer and SuperScript III reverse transcriptase (ThermoFisher Scientific, Grand Island, NY). Real-time PCR was performed with a Bio-Rad CFX384 Real-time PCR system and iQ SYBR Green Supermix (Bio-rad, Hercules, CA). 18S was measured as internal control for normalization. The comparative CT (Ct) method was used to calculate the relative mRNA expression with the average control value set as “1.” The sequences of mouse primers used in the real-time PCR process are shown in Table 1.

TABLE 1
Mouse real-time PCR primers

| Gene | | SEQ | | SEQ ID |
|---|---|---|---|---|
| name | Forward primer | ID NO: | Reverse Primer | NO: |
| 18S | GAGCGAAAGCATTTGCCAAG | 1 | GGCATCGTTTATGGTCGGAA | 2 |
| COL1A1 | GCTCCTCTTAGGGGCCACT | 3 | CCACGTCTCACCATTGGGG | 4 |
| TIMP1 | GCAACTCGGACCTGGTCATAA | 5 | CGGCCCGTGATGAGAAACT | 6 |
| SHP | TGGGTCCCAAGGAGTATGC | 7 | GCTCCAAGACTTCACACAGTG | 8 |
| MCP1 | TTAAAAACCTGGATCGGAACCAA | 9 | GCATTAGCTTCAGATTTACGGGT | 10 |
| TNFA | CCCTCACACTCAGATCATCTTCT | 11 | GCTACGACGTGGGCTACAG | 12 |
| IL1B | GCAACTGTTCCTGAACTCAACT | 13 | ATCTTTTGGGGTCCGTCAACT | 14 |
| IL6 | TAGTCCTTCCTACCCCAATTTCC | 15 | TTGGTCCTTAGCCACTCCTTC | 16 |
| CYP7A1 | GGGATTGCTGTGGTAGTGAGC | 17 | GGTATGGAATCAACCCGTTGTC | 18 |
| CYP8B1 | CCTCTGGACAAGGGTTTTGTG | 19 | GCACCGTGAAGACATCCCC | 20 |
| ASBT | GTCTGTCCCCCAAATGCAACT | 21 | CACCCCATAGAAAACATCACCA | 22 |
| FGF15 | ATGGCGAGAAAGTGGAACGG | 23 | CTGACACAGACTGGGATTGCT | 24 |

### Statistical Analysis

All results were expressed as mean±SEM. An unpaired Student's t-test was used to calculate the p value. A p<0.05 was considered statistically significant.

### Results

### G-β-MCA Treatment Attenuates Ductular Reaction and Liver Fibrosis in Cyp2c70 KO Mice

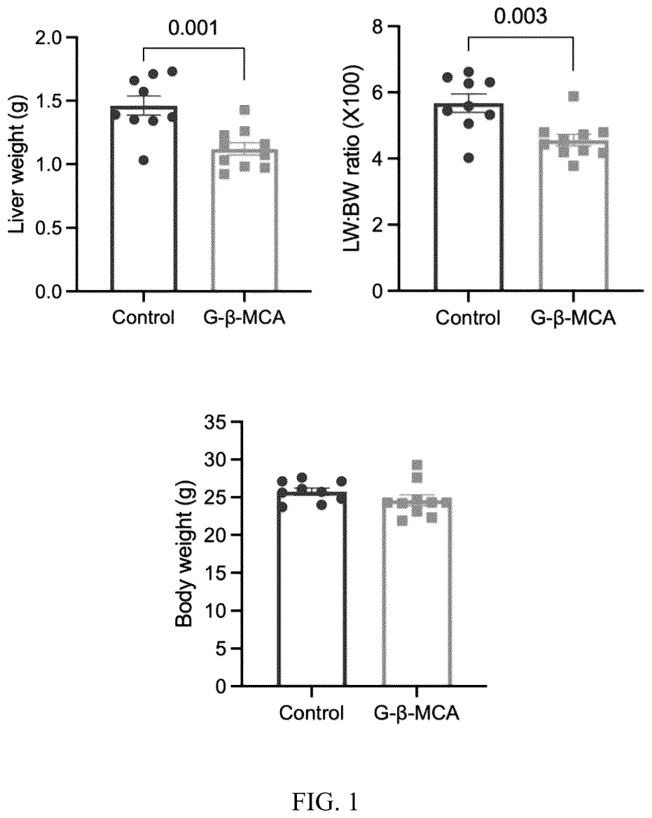

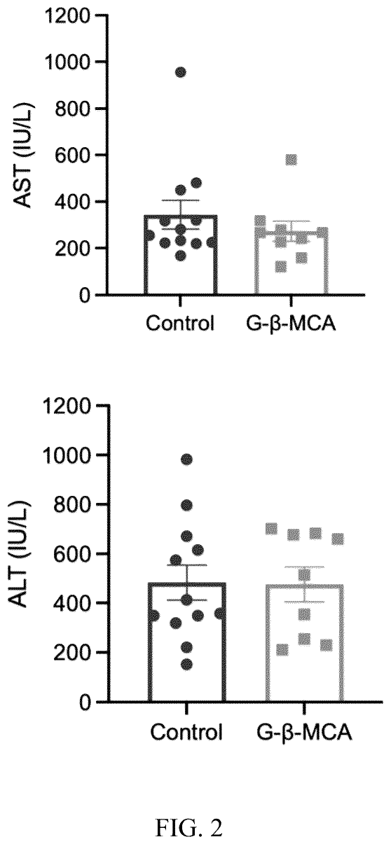

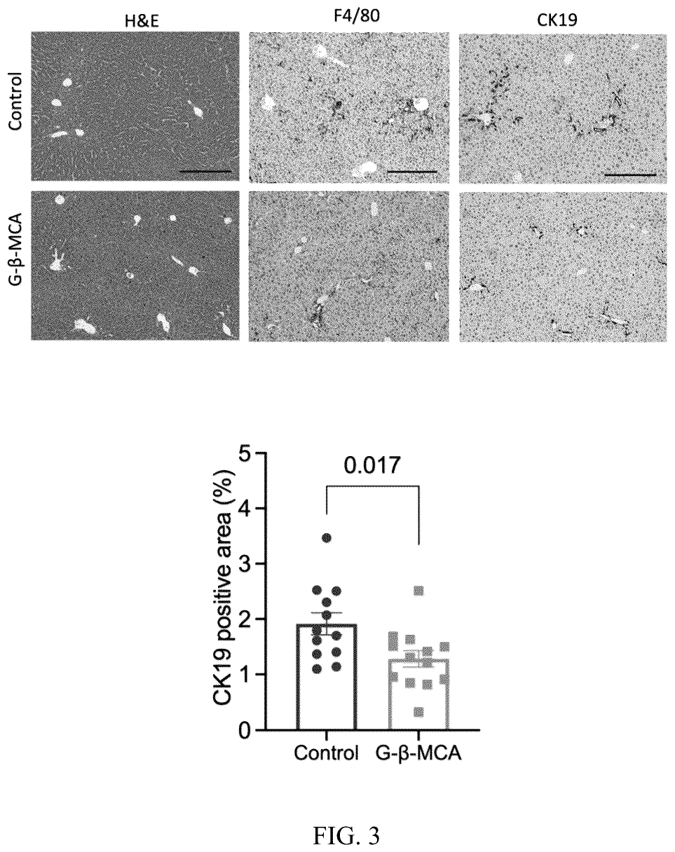

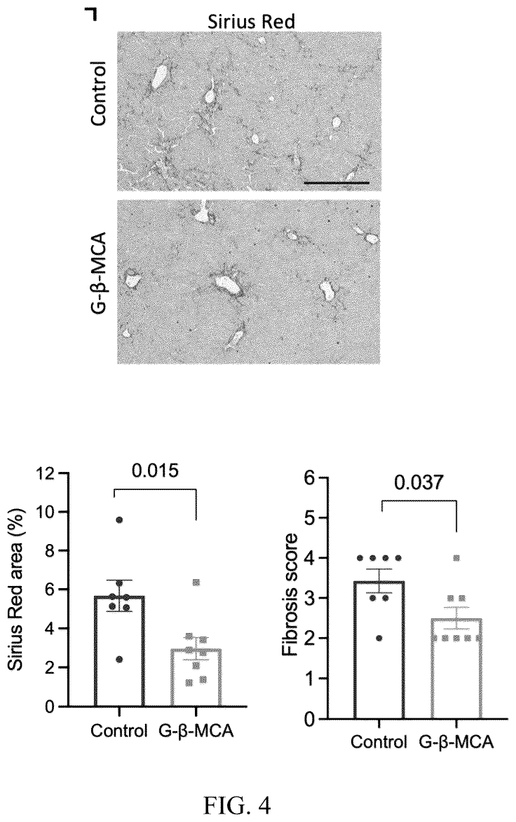

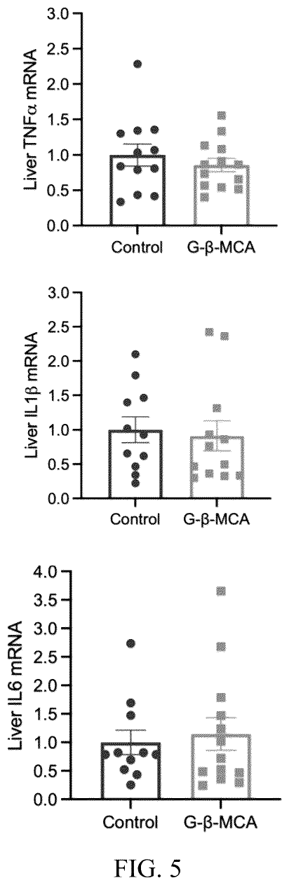

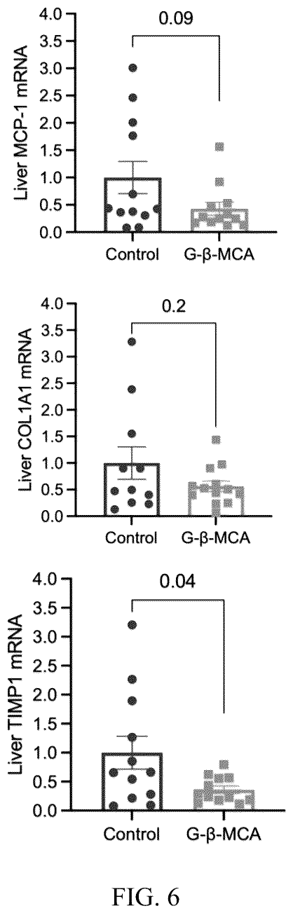

To investigate the potential anti-cholestasis effect of G-β-MCA, we treated ˜8 weeks old male Cyp2c70 KO mice with G-β-MCA for 5 weeks. G-β-MCA treatment did not affect body weight (FIG. 1, lower panel), but significantly decreased liver weight (FIG. 1, upper left panel) and liver weight to body weight ratio (FIG. 1, upper right panel). Previous work showed that Cyp2c70 KO mice have an increased liver weight to body weight ratio than WT mice, possibly due to chronic liver injury. Plasma transaminases aspartate aminotransferase (AST) and alanine aminotransferase (ALT) concentration was not significantly altered by the G-β-MCA treatment (FIG. 2). Liver histological analysis revealed that Cyp2c70 KO mice showed portal inflammation (F4/80 stain) and ductular reaction (CK-19 stain) (FIG. 3, upper panel) and liver fibrosis (Sirius red stain) (FIG. 4, upper panel and lower left panel). We did not observe apparent reduction of portal inflammatory infiltration upon G-β-MCA treatment. However, we found a significant reduction of CK19 positive stain upon G-β-MCA treatment (FIG. 3, lower panel). In addition, liver fibrosis was significantly attenuated by G-β-MCA treatment (FIG. 4, lower right panel). Analysis of gene expression markers showed that G-β-MCA did not lower liver mRNA of cytokine tumor necrosis factor α (TNFα) (FIG. 5, upper panel), interleukin 1β (IL1β) (FIG. 5, center panel), or interleukin 6 (IL6) (FIG. 5, lower panel)). The mRNA of chemokine monocyte chemoattractant protein-1 (MCP-1) and fibrosis genes collagen 1A1 (COL1A1) trended lower but did not reach statistical significance (FIG. 6, upper and center panels) and metallopeptidase Inhibitor 1 (TIMP1) (FIG. 6, lower panel) was significantly reduced in the G-β-MCA treatment group, which was consistent with reduction of liver fibrosis in G-β-MCA treated mice (FIG. 4).

### G-β-MCA Treatment Improved Gut Barrier Function in Cyp2c70 KO Mice

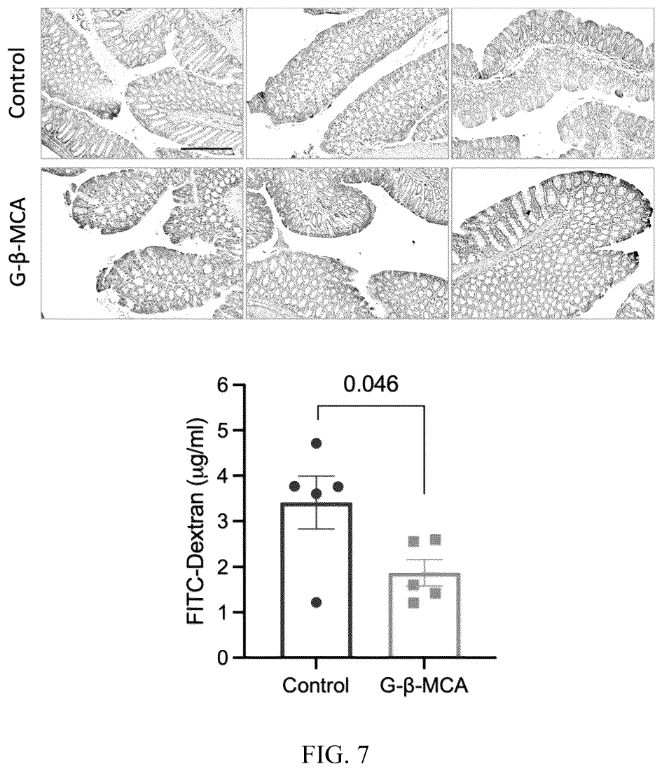

Impaired gut barrier function is known to contribute to liver inflammation and injury via the gut-liver axis. Studies from us and other have shown that Cyp2c70 KO mice show impaired gut barrier function due to hydrophobic bile acid exposure. A previous study showed that impaired gut integrity was reflected in marked reduction of Zonula Occludens-1 (ZO-1) staining in the colon epithelial cells. Here we found that mice treated with G-β-MCA appeared to show higher ZO-1 intensity in the colon epithelial cells (FIG. 7, upper panel), suggesting that G-β-MCA improves gut barrier integrity in Cyp2c70 KO mice. To obtain further functional evidence to support this effect, we further tested the effect of G-β-MCA on gut permeability by orally administering FITC-Dextran to mice. Indeed, blood FITC-Dextran concentration was significantly lower in G-β-MCA-treated Cyp2c70 KO mice than controls (FIG. 7, lower panel), supporting that G-β-MCA treatment improved gut barrier function in Cyp2c70 KO mice.

### G-β-MCA Treatment Reduced Total Bile Acid Pool Size and Biliary Bile Acid Hydrophobicity

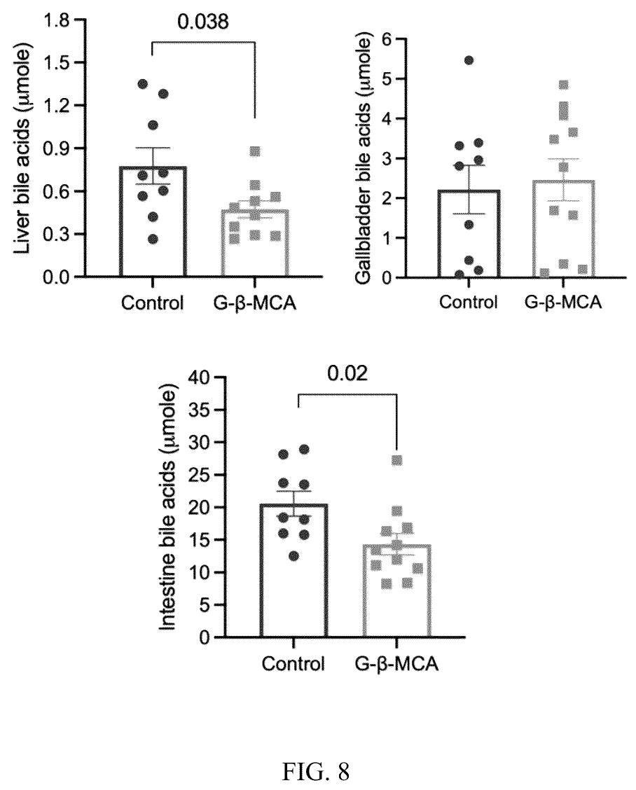

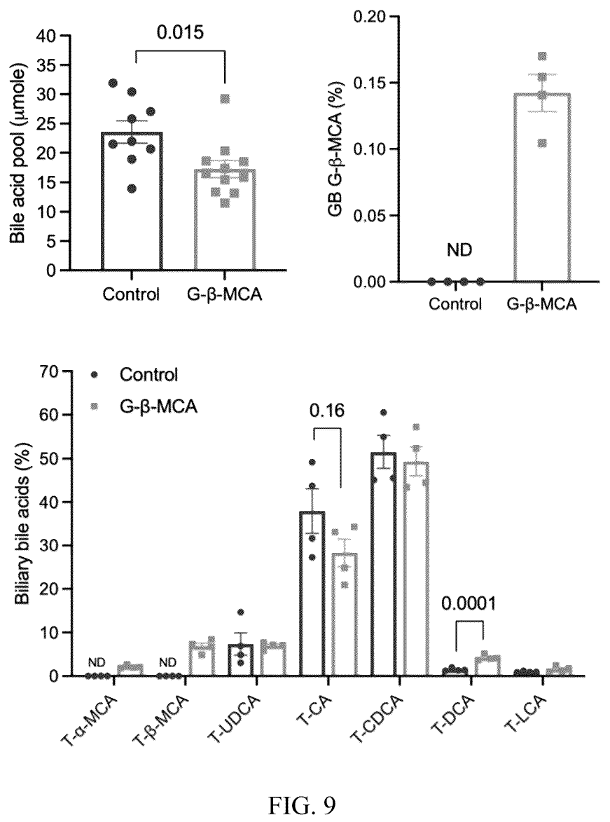

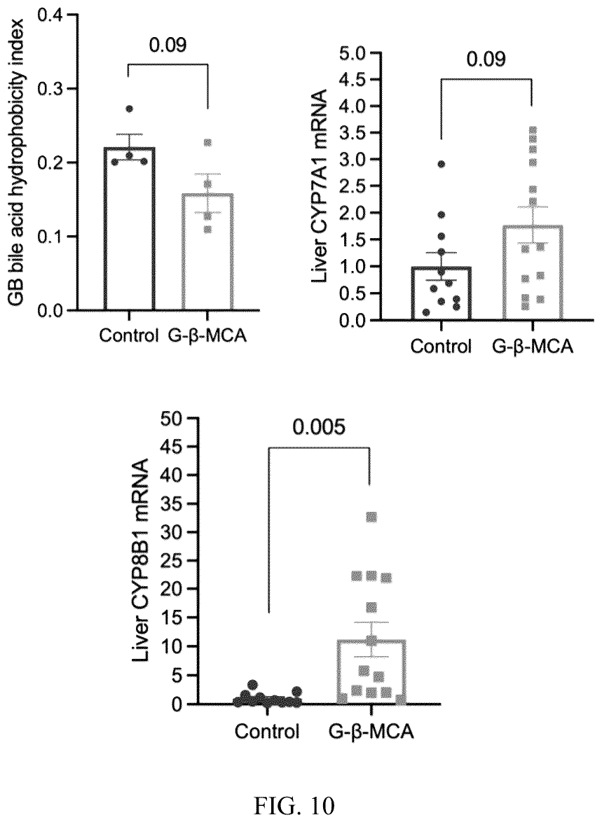

To understand how G-β-MCA treatment results in these beneficial effects in Cyp2c70 KO mice, we next analyzed how G-β-MCA treatment modulated bile acid metabolism. Unexpectedly, despite being orally administered exogenous bile acids, we found that G-β-MCA treatment significantly lowered total liver bile acids (FIG. 8, upper left panel) and small intestine bile acids (FIG. 8, lower panel) but not gallbladder total bile acids (FIG. 8, upper right panel), which was translated into a ˜27% smaller bile acid pool than untreated controls (FIG. 9, upper left panel). Interestingly, analysis of bile acid composition revealed that G-β-MCA was detectable only in the G-β-MCA-treated mice but accounted for merely less than 0.2% of the total biliary bile acids (FIG. 9, upper right panel and lower panel). In contrast, biliary bile of the G-β-MCA-treated mice was enriched with T-αMCA and T-βMCA, which accounted for about ˜10% of the total biliary bile acids, while T-αMCA and T-βMCA were not detectable in control mice (FIG. 9, upper right panel and lower panel). Given that Cyp2c70 KO mice did not produce endogenous MCA, T-αMCA and T-β-MCA detected in the bile of the treated mice was likely derived from the exogenously administered G-β-MCA. G-β-MCA treatment did not alter the relative abundance of T-UDCA, T-CDCA or T-LCA, but lowed T-CA abundance and increased T-DCA abundance (FIG. 9, upper right panel and lower panel). As a result of the presence of T-α-MCA and T-βMCA, the calculated hydrophobicity index was lower in G-β-MCA treated mice although the difference did not reach statistical significance (FIG. 10). Consistent with reduced bile acid pool size and biliary bile acid hydrophobicity, G-β-MCA treatment also increased hepatic CYP7A1 and CYP8B1 mRNA expression (FIG. 10, upper left and right panels), suggesting alleviated bile acid repression in these genes.

### G-β-MCA Treatment Promotes Fecal Bile Acid Excretion and Reduces Gut Exposure to Hydrophobic Acid

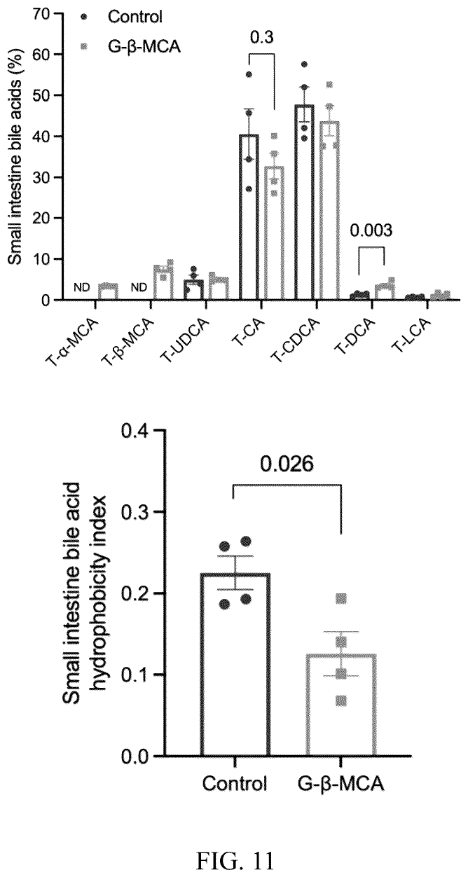

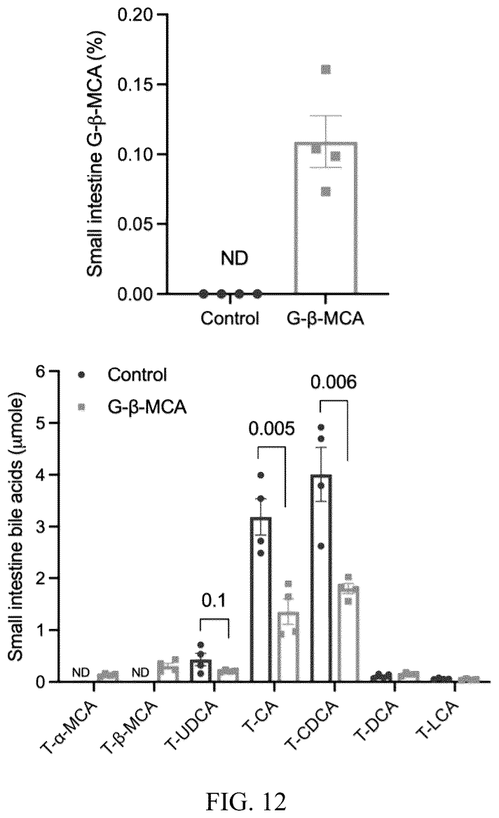

To gain a better understand of how G-β-MCA treatment modulates bile acid metabolism, we next analyzed small intestine bile acid composition. We found that the bile acid composition in the small intestine was similar to that of biliary bile acids, with T-αMCA and T-βMCA accounting for ˜10% of total bile acids, slightly lower T-CA abundance, and significantly higher T-DCA abundance in the G-β-MCA-treated mice (FIG. 11), leading to significantly reduced bile acid hydrophobicity index (FIG. 11). G-β-MCA was only detected in the treated mice but accounted for ˜0.1% of total bile acids in the small intestine (FIG. 12). As such, the amount of T-αMCA and T-βMCA is ˜100 fold higher than G-β-MCA in the small intestine of the treated Cyp2c70 KO mice. Unconjugated bile acids were largely undetectable in small intestine of either group, suggesting that bacterial enzyme-mediated bile acid deconjugation primarily occurred in the large intestine. The absolute amount of T-CA and T-CDCA was reduced in the G-β-MCA-treated mice (FIG. 12), which accounted for the significant reduction of total bile acids in the small intestine in these mice (FIGS. 8-9).

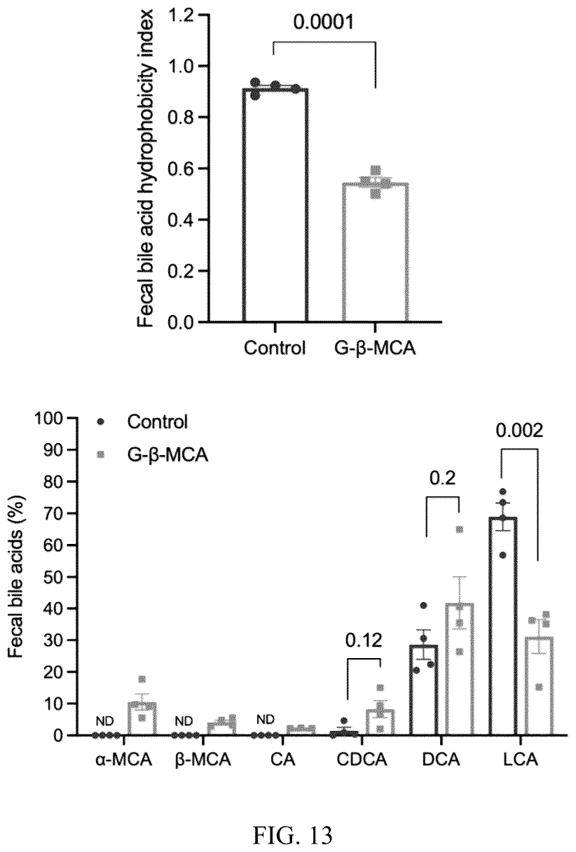

Because G-β-MCA treatment improved gut barrier function (FIG. 7), we next analyzed fecal bile acids, which has been shown to closely reflect bile acid composition in the large intestine of Cyp2c70 KO mice. Fecal bile acids are almost exclusively in the unconjugated forms and conjugated bile acids, including exogenously administered G-β-MCA, were essentially undetectable. Generally consistent with our previous published results, the fecal bile acids of Cyp2c70 KO controls mice consisted predominantly of highly hydrophobic LCA (˜70%) and, to less extent, DCA (˜30%) and only trace amount of other bile acids (FIG. 13). While αMCA and βMCA were absent in Cyp2c70 KO controls as expected, the total amount of αMCA and βMCA accounted for ˜15% of total fecal bile acids in the G-β-MCA-treated mice (FIG. 13). Furthermore, LCA abundance decreased to ˜30% while DCA and CDCA abundance were modestly higher in the G-β-MCA-treated mice (FIG. 13). Because LCA has the highest hydrophobicity index among all measured bile acids, a significant reduction of LCA abundance correlated with a significantly lower fecal bile acid hydrophobicity index in the G-β-MCA-treated mice (FIG. 13).

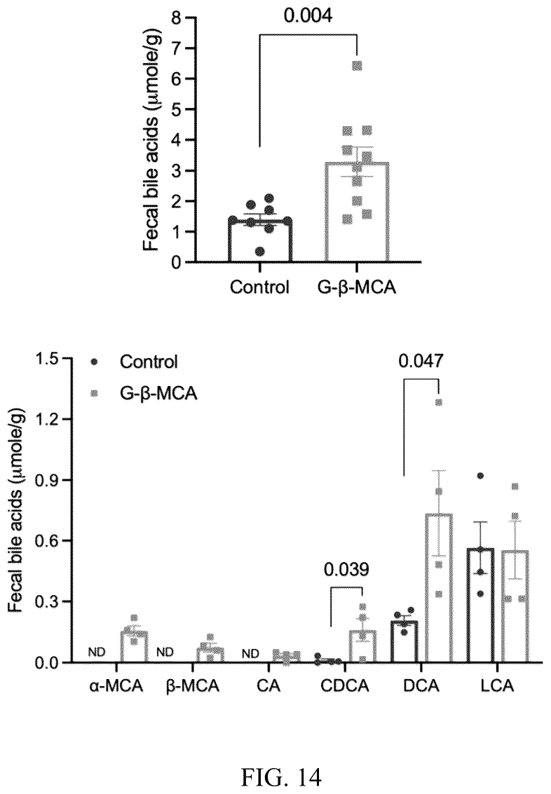

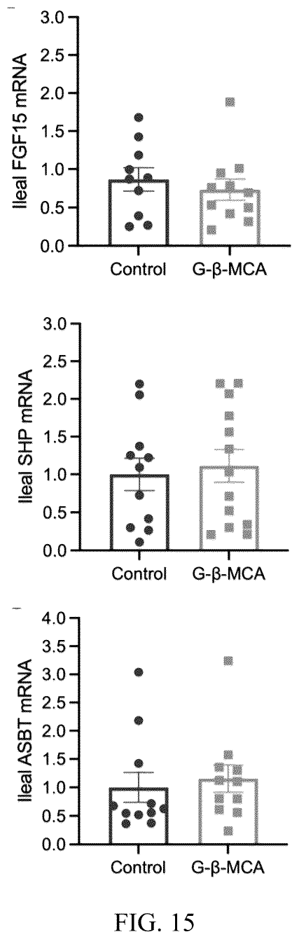

Furthermore, the G-β-MCA-treated mice showed a ˜2-fold higher fecal bile acid content, suggesting that G-β-MCA-treated mice had increased fecal bile acid excretion (FIG. 14), which may explain the reduced small intestine bile acid content and total bile acid pool in these mice (FIGS. 8-9). G-β-MCA treatment did not alter the absolute amount of LCA but significantly increased fecal CDCA and DCA, which, together with αMCA and βMCA, accounted for the overall increased fecal bile acid excretion (FIG. 14). Gene expression analysis found that G-β-MCA treatment did not alter ileal mRNA expression of FXR target gene fibroblast growth factor 15 (FGF15) or small heterodimer partner (SHP) (FIG. 15, upper and center panels). G-β-MCA treatment did not alter the mRNA of ileal bile acid uptake transporter ASBT (FIG. 15, lower panel), indicating that increased fecal bile acid excretion was not a result of downregulation of ileal ASBT expression. In summary, these results show that G-β-MCA treatment promotes fecal bile acid excretion and reduces the gut bile acid pool hydrophobicity.

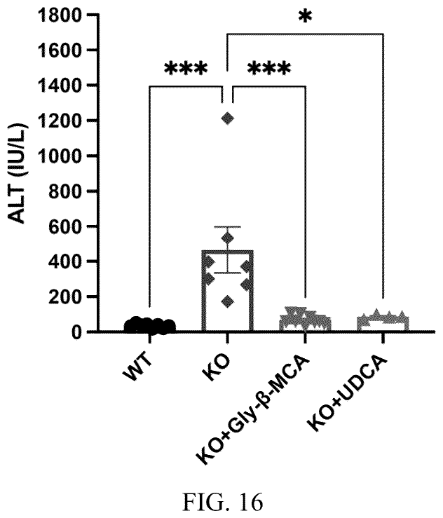

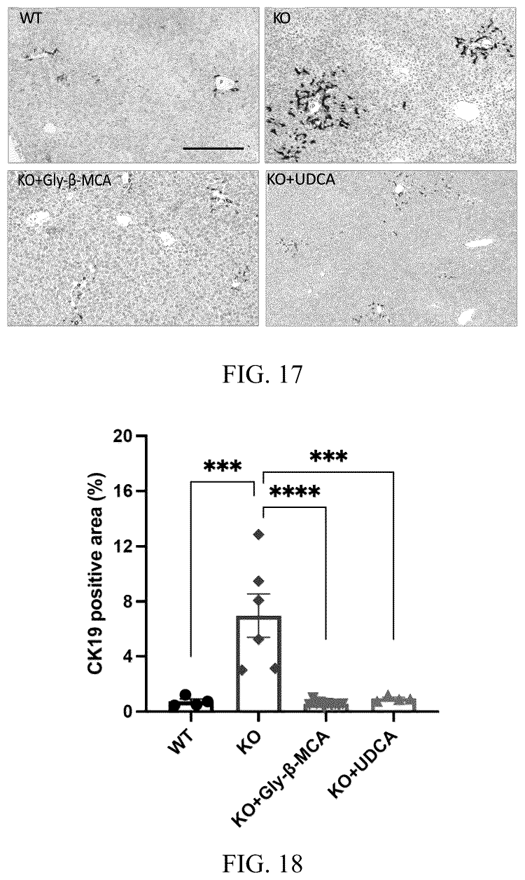

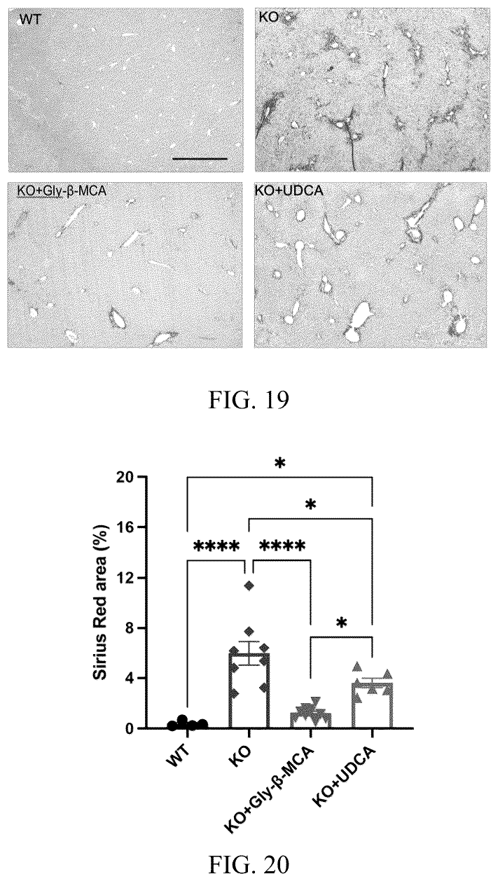

As noted above, currently UDCA remains the first line treatment for many forms of cholestasis in humans. However, high dose of UDCA can also increase lithocholic acid (LCA) production, which contributes to treatment-associated adverse events. Results provided in FIGS. 16-20 demonstrate that Gly-β-MCA provided at dosages of 160 mg/kg per day reduces liver injury as well or better than UDCA. FIG. 16 shows that Gly-β-MCA and UDCA treatment reduce a liver injury marker serum alanine aminotransferase (ALT) in female Cyp2c70 KO mice. FIGS. 17-18 shows that Gly-β-MCA and UDCA treatment reduce ductular reaction in female Cyp2c70 KO mice. FIGS. 19-20 shows that Gly-β-MCA and UDCA treatment reduce portal fibrosis in female Cyp2c70 KO mice. Gly-β-MCA performs significantly better than UDCA.

### DISCUSSION

Cyp2c70 KO mice show a “human-like” hydrophobic bile acid pool and have become a useful model for study of bile acid-induced injury in cholestasis. In this study, we investigated the potential beneficial effect of G-β-MCA treatment in Cyp2c70 KO mice based on its hydrophilic physiochemical property and signaling property as an FXR antagonist. We have found that G-β-MCA treatment reduced total and hepatic bile acid pool size and biliary bile acid hydrophobicity. These changes did not improve markers of liver inflammation but attenuated ductular reaction and liver fibrosis. In addition, G-β-MCA treatment improved gut barrier function, which is attributed to reduced gut bile acid hydrophobicity despite increased fecal bile acid excretion. The absence of endogenous MCAs in Cyp2c70 KO mice allowed us to obtain better understanding of how G-β-MCA treatment modulated bile acid metabolism to account for the observed therapeutic benefits, which is further discussed below.

G-β-MCA was previously shown to be poorly absorbed in the small intestine and therefore acted as a gut-restricted FXR inhibitor. In addition, it was shown that G-β-MCA was more resistant to the hydrolysis by bacterial bile salt hydrolase when compared to the mouse endogenous T-β-MCA. Unexpectedly, our study detected very low levels of G-β-MCA not only in bile but also in small intestine and feces of the G-β-MCA-treated Cyp2c70 KO mice. Instead, T-αMCA and T-βMCA were detected in the bile and small intestine and αMCA and βMCA were detected in the feces of the G-β-MCA-treated Cyp2c70 KO mice. If G-β-MCA can be efficiently absorbed in the terminal ileum like other endogenous bile acids, then a higher amount of G-β-MCA than T-MCAs is expected to be present in the bile of the G-β-MCA treated mice. However, we found that the total biliary concentration of T-α-MCA and T-β-MCA was about 60 times higher than biliary G-β-MCA concentration. An explanation for our observation was that G-β-MCA was poorly absorbed in the ileum and most G-β-MCA reached the large intestine where it was deconjugated to β-MCA, some of which was further epimerized by bacterial enzymes to α-MCA. Both α-MCA and β-MCA can be passively absorbed in the large intestine and transported to the liver where they were conjugated to become T-α-MCA and T-β-MCA.

In a previous study, G-β-MCA hydrolysis by bacterial bile salt hydrolase was tested in fecal protein solution in a 20-minute in vitro reaction and only T-β-MCA, but not G-β-MCA, was found to be rapidly deconjugated. Similarly, our recent study also showed rapid deconjugation of T-CDCA in mouse fecal slurry in vitro. These data suggest that bacterial bile salt hydrolase may show slower kinetics toward G-β-MCA than taurine-conjugated bile acids. Nevertheless, our data shows that G-β-MCA can still be efficiently deconjugated during colonic transit under in vivo condition. The small intestine contained predominantly conjugated bile acids and essentially undetectable unconjugated bile acids, suggesting that quantitatively significant deconjugation of G-β-MCA, after oral intake, only occurred after G-β-MCA reached the large intestine. However, we detected extremely low amount of G-β-MCA in the small intestine. This is because we subjected mice to a 6-hour fast before tissues were collected. Since the entire gastrointestinal transit time and small intestine transit time in mice were reported to be about 6-8 hours and about 2-3 hours, respectively, a 6 hour fast was sufficient for most orally acquired G-β-MCA to enter the large intestine to be deconjugated in our study, which provides an explanation of the low G-β-MCA level detected in the small intestine of the G-β-MCA treated mice. As such, orally administered G-β-MCA acts as FXR antagonist in the small intestine and is subsequently converted to T-MCAs to reduce biliary bile acid pool hydrophobicity.

In early experiments in the present work, the daily G-β-MCA intake per mouse was ˜1 μmole/day compared to the total endogenous bile acid pool of 15-20 μmole in Cyp2c70 KO mice. If the exogenous G-β-MCA is efficiently preserved in the enterohepatic circulation, G-β-MCA treatment over 5 weeks is expected to significantly expand the total bile acid pool in Cyp2c70 KO mice. On the contrary, G-β-MCA treatment significantly reduced total bile acid pool of Cyp2c70 KO mice, which is attributed to significantly increased fecal bile acid excretion. Interestingly, in addition to fecal α-MCA and β-MCA excretion, we found that the G-β-MCA-treated mice showed significantly increased fecal excretion of DCA and CDCA, but not LCA. Consistently, we found that T-CA and T-CDCA in the small intestine were significantly lower in the G-β-MCA-treated mice. Efficient conversion of T-CA to DCA in the large intestine can explain that DCA and, to much less extent, CA were enriched in the feces of the G-β-MCA-treated mice. In contrast, T-CDCA did not drive further increase of fecal LCA enrichment, and therefore fecal CDCA was significantly increased in the G-β-MCA-treated mice. These changes showed that intestine bile acid absorption was reduced in the G-β-MCA-treated mice. In contrast, reduced liver bile acids and unaltered gallbladder bile acids did not show that reduced biliary bile acid secretion accounted for lower small intestine bile acids. G-β-MCA treatment did not reduce ileal ASBT expression.

In this work, we found that reduced bile acid pool size and hydrophobicity correlated with modestly elevated hepatic CYP7A1 mRNA expression and a more robust induction of hepatic CYP8B1 mRNA, which was consistent with bile acid feedback inhibition of bile acid synthesis genes. Increased hepatic CYP8B1 explains the increased production of T-CA which was subsequently converted to T-DCA in the G-β-MCA-treated mice. We have also reported liver CYP8B1 induction and increased T-DCA abundance by ASBT inhibitor treatment in Cyp2c70 KO mice, suggesting these changes are a common response to blocked intestine bile acid uptake. However, we did not see reduced ileal SHP or FGF15 expression in the G-β-MCA-treated mice, which was unexpected since total bile acid amount was reduced and FXR antagonists T-MCAs were enriched in the small intestine of these mice. It should be noted that bile acid signaling in the enterohepatic circulation can be significantly altered at baseline in Cyp2c70 KO mice compared to WT mice due at least in part to altered bile acid composition and the presence of inflammation and injury. These changes can underlie differential responses to stimuli in Cyp2c70 KO mice and WT mice.

In the work described in the present disclosure, we initially chose ˜20 mg/kg G-β-MCA daily dose, which was within previously tested dosing range in obese mice. At this dosage level, G-β-MCA treatment reduced bile acid pool size and hydrophobicity, which contributes to alleviated liver fibrosis and improved gut barrier function in Cyp2c70 KO mice. However, G-β-MCA treatment at ˜20 mg/kg per daily dose did not cause robust reduction of hepatic inflammation and injury markers, which could be because biliary MCA enrichment was relatively modest. However, G-β-MCA, when dosed at an 8× greater level of 160 mg/kg per day, hepatic inflammation and injury markers were reduced at least as well as UDCA. As noted, currently, UDCA remains the first line treatment of many forms of cholestasis in humans. However, high doses of UDCA can also increase LCA production contributing to treatment associated adverse events.

While the present disclosure has been described herein in connection with certain embodiments so that aspects thereof may be more fully understood and appreciated, it is not intended that the present disclosure be limited to these particular embodiments. On the contrary, it is intended that all alternatives, modifications, and equivalents are included within the scope of the present disclosure as defined herein. Thus the embodiments described above, which include particular embodiments, will serve to illustrate the practice of the inventive concepts of the present disclosure, it being understood that the particulars shown are by way of example and for purposes of illustrative discussion of particular embodiments only and are presented in the cause of providing what is believed to be the most useful and readily understood description of methods and procedures as well as of the principles and conceptual aspects of the present disclosure. Changes may be made in the formulations of the various compositions described herein, the methods described herein or in the steps or the sequence of steps of the methods described herein without departing from the spirit and scope of the present disclosure. Further, while various embodiments of the present disclosure have been described in exemplary claims herein below, it is not intended that the present disclosure be limited to these particular exemplary claims.

What is claimed is:

1. A method of treating a cholestasis liver condition in a human subject in need of such therapy, comprising:
administering to the human subject a muricholic acid (MCA) or a pharmaceutically acceptable salt thereof, and wherein the treatment with the MCA reduces total and hepatic bile acid pool size and biliary bile acid hydrophobicity in the liver of the human subject.

2. The method of claim 1, wherein the cholestasis liver condition is at least one of (1) Primary biliary cholangitis, (2) Primary sclerosing cholangitis, (3) Biliary atresia, (4) Progressive familial intrahepatic cholestasis 1 (PFIC 1), (5) Progressive familial intrahepatic cholestasis 2 (PFIC 2), (6) Progressive familial intrahepatic cholestasis 3 (PFIC 3), and (7) Progressive familial intrahepatic cholestasis 4 (PFIC 4).

3. The method of claim 1, wherein the MCA is selected from the group consisting of α-MCA, β-MCA, ω-MCA, glycine-conjugated α-MCA (G-α-MCA), glycine-conjugated β-MCA (G-β-MCA), glycine-conjugated ω-MCA (G-ω-MCA), taurine-conjugated α-MCA (T-α-MCA), taurine-conjugated β-MCA (T-β-MCA), and taurine-conjugated ω-MCA (T-ω-MCA).

4. The method of claim 3, wherein the cholestasis liver condition is at least one of (1) Primary biliary cholangitis, (2) Primary sclerosing cholangitis, (3) Biliary atresia, (4) Progressive familial intrahepatic cholestasis 1 (PFIC 1), (5) Progressive familial intrahepatic cholestasis 2 (PFIC 2), (6) Progressive familial intrahepatic cholestasis 3 (PFIC 3), and (7) Progressive familial intrahepatic cholestasis 4 (PFIC 4).

5. A method of treating a cholestasis liver condition in a human subject in need of such therapy, comprising:
administering to the human subject a glycine- or taurine-conjugated muricholic acid (MCA) or a pharmaceutically acceptable salt thereof, and wherein the treatment with the glycine- or taurine-conjugated MCA reduces total and hepatic bile acid pool size and biliary bile acid hydrophobicity in the liver of the human subject.

6. The method of claim 5, wherein the cholestasis liver condition is at least one of (1) Primary biliary cholangitis, (2) Primary sclerosing cholangitis, (3) Biliary atresia, (4) Progressive familial intrahepatic cholestasis 1 (PFIC 1), (5) Progressive familial intrahepatic cholestasis 2 (PFIC 2), (6) Progressive familial intrahepatic cholestasis 3 (PFIC 3), and (7) Progressive familial intrahepatic cholestasis 4 (PFIC 4).

7. The method of claim 5, wherein the glycine- or taurine-conjugated MCA is selected from the group consisting of glycine-conjugated α-MCA (G-α-MCA), glycine-conjugated β-MCA (G-β-MCA), glycine-conjugated ω-MCA (G-ω-MCA), taurine-conjugated α-MCA (T-α-MCA), taurine-conjugated β-MCA (T-β-MCA), and taurine-conjugated ω-MCA (T-ω-MCA).

8. The method of claim 7, wherein the cholestasis liver condition is at least one of (1) Primary biliary cholangitis, (2) Primary sclerosing cholangitis, (3) Biliary atresia, (4) Progressive familial intrahepatic cholestasis 1 (PFIC 1), (5) Progressive familial intrahepatic cholestasis 2 (PFIC 2), (6) Progressive familial intrahepatic cholestasis 3 (PFIC 3), and (7) Progressive familial intrahepatic cholestasis 4 (PFIC 4).

9. A method of treating a cholestasis liver condition in a human subject in need of such therapy, comprising:
administering to the human subject a glycine- or taurine-conjugated muricholic acid (MCA) selected from a glycine-conjugated β-MCA (G-β-MCA), or a taurine-conjugated β-MCA (T-β-MCA), or a pharmaceutically acceptable salt thereof, and wherein the treatment with the G-β-MCA, T-β-MCA, or pharmaceutically acceptable salt thereof reduces total and hepatic bile acid pool size and biliary bile acid hydrophobicity in the liver of the human subject.

10. The method of claim 9, wherein the cholestasis liver condition is at least one of (1) Primary biliary cholangitis, (2) Primary sclerosing cholangitis, (3) Biliary atresia, (4) Progressive familial intrahepatic cholestasis 1 (PFIC 1), (5) Progressive familial intrahepatic cholestasis 2 (PFIC 2), (6) Progressive familial intrahepatic cholestasis 3 (PFIC 3), and (7) Progressive familial intrahepatic cholestasis 4 (PFIC 4).

11. The method of claim 9, wherein the glycine- or taurine-conjugated MCA is glycine-conjugated β-MCA.

12. The method of claim 9, wherein the glycine- or taurine-conjugated MCA is taurine-conjugated β-MCA.


SEQUENCE LISTING (24 sequences)

SEQ ID NO: 1 - DNA, 20 bp, Mus musculus
  source 1..20: mol_type = genomic DNA
gagcgaaagcatttgccaag

SEQ ID NO: 2 - DNA, 20 bp, Mus musculus
  source 1..20: mol_type = genomic DNA
ggcatcgtttatggtcggaa

SEQ ID NO: 3 - DNA, 19 bp, Mus musculus
  source 1..19: mol_type = genomic DNA
gctcctcttaggggccact

SEQ ID NO: 4 - DNA, 19 bp, Mus musculus
  source 1..19: mol_type = genomic DNA
ccacgtctcaccattgggg

SEQ ID NO: 5 - DNA, 21 bp, Mus musculus
  source 1..21: mol_type = genomic DNA
gcaactcggacctggtcataa

SEQ ID NO: 6 - DNA, 19 bp, Mus musculus
  source 1..19: mol_type = genomic DNA
cggcccgtgatgagaaact

SEQ ID NO: 7 - DNA, 19 bp, Mus musculus
  source 1..19: mol_type = genomic DNA
tgggtcccaaggagtatgc

SEQ ID NO: 8 - DNA, 21 bp, Mus musculus
  source 1..21: mol_type = genomic DNA
gctccaagacttcacacagtg

SEQ ID NO: 9 - DNA, 23 bp, Mus musculus
  source 1..23: mol_type = genomic DNA
ttaaaaacctggatcggaaccaa

SEQ ID NO: 10 - DNA, 23 bp, Mus musculus
  source 1..23: mol_type = genomic DNA
gcattagcttcagatttacgggt

SEQ ID NO: 11 - DNA, 23 bp, Mus musculus
  source 1..23: mol_type = genomic DNA
ccctcacactcagatcatcttct

SEQ ID NO: 12 - DNA, 19 bp, Mus musculus
  source 1..19: mol_type = genomic DNA
gctacgacgtgggctacag

SEQ ID NO: 13 - DNA, 22 bp, Mus musculus
  source 1..22: mol_type = genomic DNA
gcaactgttcctgaactcaact

SEQ ID NO: 14 - DNA, 21 bp, Mus musculus
  source 1..21: mol_type = genomic DNA
atcttttggggtccgtcaact

SEQ ID NO: 15 - DNA, 23 bp, Mus musculus
  source 1..23: mol_type = genomic DNA
tagtccttcctaccccaatttcc

SEQ ID NO: 16 - DNA, 21 bp, Mus musculus
  source 1..21: mol_type = genomic DNA
ttggtccttagccactccttc

SEQ ID NO: 17 - DNA, 21 bp, Mus musculus
  source 1..21: mol_type = genomic DNA
gggattgctgtggtagtgagc

SEQ ID NO: 18 - DNA, 22 bp, Mus musculus
  source 1..22: mol_type = genomic DNA
ggtatggaatcaacccgttgtc

SEQ ID NO: 19 - DNA, 21 bp, Mus musculus
  source 1..21: mol_type = genomic DNA
cctctggacaagggttttgtg

SEQ ID NO: 20 - DNA, 19 bp, Mus musculus
  source 1..19: mol_type = genomic DNA
gcaccgtgaagacatcccc

SEQ ID NO: 21 - DNA, 21 bp, Mus musculus
  source 1..21: mol_type = genomic DNA
gtctgtcccccaaatgcaact

SEQ ID NO: 22 - DNA, 22 bp, Mus musculus
  source 1..22: mol_type = genomic DNA
caccccatagaaaacatcacca

SEQ ID NO: 23 - DNA, 20 bp, Mus musculus
  source 1..20: mol_type = genomic DNA
atggcgagaaagtggaacgg

SEQ ID NO: 24 - DNA, 21 bp, Mus musculus
  source 1..21: mol_type = genomic DNA
ctgacacagactgggattgct